(19) United States
(12) Patent Application Publication
(10) Pub. No.: US 2002/0009439 A1
(43) Pub. Date: Jan. 24, 2002

(54) Compounds effecting neuron remodeling and assays for same

(76) Inventors: Robert W. Mahley, San Francisco, CA (US); Karl H. Weisgraber, Walnut Creek, CA (US); Robert E. Pitas, Albany, CA (US)

Correspondence Address: Paula A. Borden, BOZICEVIC, FIELD & FRANCIS LLP, 200 Middlefield Road, Suite 200, Menlo Park, CA, 94025, US

(21) Appl. No.: 09/782,757

(22) Filed: Feb. 12, 2001

Related U.S. Application Data
(63) Continuation-in-part of application No. 09/070,675, filed on Apr. 30, 1998, now abandoned.
Application No. 09/070,675 is a continuation-in-part of application No. 08/659,785, filed on Jan. 19, 1996, now abandoned.
(60) Provisional application No. 60/005,550, filed on Oct. 17, 1995.

(51) Int. Cl. A61K 31/00; A61K 39/395

(52) U.S. Cl. 424/130.1; 514/1

(57) ABSTRACT

Compounds, compositions and therapeutic methods are disclosed for the treatment of the central nervous system. The compounds are derived from the disclosed assay system which tests compounds for their ability to effect neuronal remodeling and neurite outgrowth. The assay uses cell cultures which have been genetically engineered to effect the expression of apoE3 and/or apoE4. A test compound is brought into contact with engineered neuronal cells in the presence of a lipid such as β-VLDL to determine the affects of the compound, if any, on the neuronal remodeling and neurite outgrowth. Compounds found to promote neurite outgrowth are used therapeutically in the treatment of diseases and/or damage to the central nervous system.

### CROSS-REFERENCES

This application is a continuation-in-part of U.S. patent application Ser. No. 09/070,675, filed Apr. 30, 1998, which is a continuation-in-part of U.S. patent application Ser. No. 08/659,785, filed Jan. 19, 1996 which is a continuation-in-part of U.S. provisional application Ser. No. 60/005,550, filed Oct. 17, 1995, each of which is hereby incorporated in its entirety herein by reference and to which applications we claim priority.

### GOVERNMENT GRANT

This invention was funded in part with funds from National Institutes of Health Program Project Grant HL41633. The Government may have certain rights to this invention.

### FIELD OF THE INVENTION

The invention relates to compounds effecting neuronal remodeling and assays for screening compounds for their effects, if any, on neuronal remodeling and neurite outgrowth. More specifically, the invention relates to cell culture assay systems wherein the cells have been genetically engineered to affect the expression of apoE3 and/or apoE4 and to compounds and treatments derived from such assays. The invention further relates to compounds that reduce apoE4 domain interaction, and methods of treating disorders related to apoE4.

### BACKGROUND OF THE INVENTION

ApoE, a 34,000 molecular weight protein is the product of a single gene on chromosome 19 and exists in three major isoforms designated apoE2, apoE3 and apoE4 for review, see Mahley (in press) in: Molecular and Genetic Bases of Neurological Disease 2nd ed.; and Mahley (1988) Science 240:622-630. The different isoforms result from amino acid substitutions at amino acid residue positions 112 and 158. The common isoform, apoE3, has a cysteine residue at position 112 and an arginine residue at position 158. The apoE4 isoform differs from apoE3 only at position 112, which is an arginine residue. The apoE2 isoform, associated with type III hyperlipoproteinemia (Mahley (1988)), differs from apoE3 only at position 158, which is a cysteine residue. ApoE3 and apoE4 bind normally to the low density lipoprotein (LDL) receptor, whereas apoE2 does not.

ApoE contains two structural domains: an amino-terminal and a carboxy-terminal domain. Weisgraber (1994) Adv. Protein Chem. 45:249-302. Each domain is associated with a specific function. The amino terminal domain contains the lipoprotein receptor binding region and the carboxy-terminal domain contains the major lipid-binding elements. The two domains appear to interact with each other in an isoform-specific manner such that amino acid substitutions in one domain influence the function of the other domain, a phenomenon referred to as domain interaction. Domain interaction is responsible for the preference of apoE4 for very low density lipoproteins (VLDL) contrasted with the preference of apoE3 for high density lipoproteins (HDL). The specific amino acid residues in apoE4 that are involved in this interaction have been identified: arginine-61 in the amino-terminal domain and glutamic acid-255 in the carboxy-terminal domain. Dong et al. (1994) J. Biol. Chem. 269:22358-22365; and Dong and Weisgraber (1996) J. Biol. Chem. 271:19053-19057.

By redistributing lipids among the cells of different organs, apoE plays a critical role in lipid metabolism. While apoE exerts this global transport mechanism in chylomicron and VLDL metabolism, it also functions in the local transport of lipids among cells within a tissue. Cells with excess cholesterol and other lipids may release these substances to apoE-lipid complexes or to HDL containing apoE, which can transport the lipids to cells requiring them for proliferation or repair. The apoE on these lipoprotein particles mediates their interaction and uptake via the LDL receptor or the LRP.

ApoE plays a neurobiological role. ApoE mRNA is abundant in the brain, where it is synthesized and secreted primarily by astrocytes. Elshourbagy et al. (1985) Proc. Natl. Acad. Sci. USA 82:203-207; Boyles et al. (1985) J. Clin. Invest. 76:1501-1513; and Pitas et al. (1987) Biochem. Biophys. Acta 917:148-161. The brain is second only to the liver in the level of apoE mRNA expression. ApoE-containing lipoproteins are found in the cerebrospinal fluid and appear to play a major role in lipid transport in the central nervous system (CNS). Pitas et al. (1987) J. Biol. Chem. 262:14352-14360. In fact, the major cerebrospinal fluid lipoprotein is an apoE-containing HDL. ApoE plus a source of lipid promotes marked neurite extension in dorsal root ganglion cells in culture. Handelmann et al. (1992) J. Lipid Res. 33:1677-1688. ApoE levels dramatically increase (about 250-fold) after peripheral nerve injury. Müiller et al. (1985) Science 228:499-501; and Ignatius et al. (1986) Proc. Natl. Acad. Sci. USA 83:1125-1129. ApoE appears to participate both in the scavenging of lipids generated after axon degeneration and in the redistribution of these lipids to sprouting neurites for axon regeneration and later to Schwann cells for remyelination of the new axons. Boyles et al. (1989) J. Clin. Invest. 83:1015-1031; and Ignatius et al. (1987) Science 236:959-962.

Most recently, apoE has been implicated in Alzheimer's disease and cognitive performance. Saunders et al. (1993) Neurol. 43:1467-1472; Corder et al. (1993) Science 261:921-923; and Reed et al. (1994) Arch. Neurol. 51:1189-1192. ApoE4 is associated with the two characteristic neuropathologic lesions of Alzheimer's disease; extracellular neuritic plaques representing deposits of amyloid beta (Aβ) peptide and intracellular neurofibrillary tangles representing filaments of hyperphosphorylated tau, a microtubule-associated protein. For review, see, McKhann et al. (1984) Neurol. 34:939-944; Selkoe (1991) Neuron 6:487 498; Crowther (1993) Curr. Opin. Struct. Biol. 3:202-206; Roses (1994) Curr. Neurol. 14:111-141; Weisgraber et al. (1994) Curr. Opin. Lipidol. 5:110-116; and Weisgraber et al. (1994) Curr. Opin. Struct. Biol. 4:507-515.

Alzheimer's disease is generally divided into three categories: early-onset familial disease (occurring before 60 years of age and linked to genes on chromosomes 21 and 14); late-onset familial disease; and sporadic late-onset disease. Both types of late-onset disease have recently been linked to chromosome 19 at the apoE locus. Other results suggest that apoE4 is directly linked to the severity of the disease in late-onset families. Roses (1994). Recently, cholesterol lowering drugs, the statins, have been suggested for use in treating Alzheimer's disease by lowering apoE4 levels. WO 95/06470.

The neurofibrillary tangles, which are paired helical filaments of hyperphosphorylated tau, accumulate in the cytoplasm of neurons. Tau is a microtubule-associated phosphoprotein which normally participates in microtubule assembly and stabilization; however, hyperphosphorylation impairs its ability to interact with microtubules. Increased binding of tau by apoE has been suggested as a treatment for Alzheimer's disease. WO 95/06456.

In vitro tau interacts with apoE3, but not with apoE4. Strittmatter et al. (1994) Exp. Neurol. 125:163-171. The interaction of apoE3 with tau may prevent its hyperphosphorylation, thus allowing it to function normally in stabilizing microtubular structure and function. In the presence of apoE4, tau could become hyperphosphorylated and thus inactive, which could promote the formation of neurofibrillary tangles.

ApoE4 has recently been associated with decreased learning ability and impaired memory. Helkala et al. (1995) Neurosci. Letts. 191:141-144. ApoE4 has been found to be a strong predictor of the outcome of patients designated as having memory impairment. Note that, apoE4 has been described as a risk factor, rather than a diagnostic. Peterson et al. (1995) JAMA 273:1274-1278; and Feskens et al. (1994) BMJ 309:1202-1206.

ApoE interacts with both the LDL receptor and the LRP and undoubtedly with other apoE-binding receptors on neurons. The LRP has been found to be increased after brain injury or glial cell conversion to neoplasia. Lopes et al. (1994) FEBS Lett. 338:301-305. The LRP was previously identified as the—macroglobulin receptor. Strickland et al. (1991) J. Biol. Chem. 266:13364-13369; and Borth (1992) FASEB J. 6:3345-3353. ApoE does not directly bind to the LRP but must first associate with cell surface heparin sulfate proteoglycans (HSPG). Mahley et al. (1991) Curr. Opin. Lipidol. 2:170-176; and Ji et al. (1994) J. Biol. Chem. 269:2764-2772. The LRP also binds a number of other ligands, including t-PA,I₂-macroglobulin-protease complex, thrombospondin-1, Pseudomonas exotoxin A, the receptor associated protein (RAP) and lactoferrin. The LRP ligand binding sites have been at least partially described. Orth et al. (1994) J. Biol. Chem. 269:21117-21122; Godyna et al. (1995) J. Cell. Biol. 129:1403-1410; Kounnas et al. (1992) J. Biol. Chem. 267:12420-12423; Willnow et al. (1994) J. Cell Sci. 107:719-726; Meilinger et al. (1995) FEBS Lett. 360:70-74; Warshawsky et al. (1993) J. Biol. Chem. 268:22046-22054; and Willnow et al. (1994) J. Biol. Chem. 269:15827-15832.

It has previously been shown that incubation of dorsal root ganglion neurons in culture with β-VLDL alters the neurite growth of these cells compared to that of cells grown in media alone. Handelmann et al. (1992). In the presence of a source of lipid (β-VLDL or free cholesterol), neurite outgrowth is greatly enhanced, specifically due to extensive branching (with little or no increased neurite extension). When the β-VLDL was enriched with exogenous rabbit apoE (equivalent to human apoE3 with respect to the occurrence of a cysteine residue at position 112) enhanced neurite extension was seen. A lipid source appears to enhance membrane biosynthesis, whereas the addition of excess rabbit apoE with a lipid source results in long neuritic extensions and a trimming back of the branches. It has also been found that the inhibitory effect of apoE4 on neurite outgrowth is associated with microtubule polymerization, whereas apoE3 supports microtubule formation. Nathan et al. (1995) J. Biol. Chem. 270:19791-19799.

Neural plasticity, maintenance of existing or formation of new synaptic connections, is critical for normal brain function, including memory. This process can be compromised by various forms of stress, including, but not limited to, age, deposition of plaques and neurofibrillary tangles in Alzheimer's disease and oxygen deprivation. Interference with neuron remodeling can lead to impaired brain function or neurodegeneration of which dementia and Alzheimer's disease are extreme examples. In the case of Alzheimer's disease alone, approximately 4 million individuals are affected in the United States. With the aging of the population, this number is projected to triple in the next twenty years. The present health care cost of Alzheimer's disease is estimated at $90 billion per year in the United States alone. Delaying the average onset of this disease for even ten years would drastically reduce the financial burdens on society and the financial and emotional burdens of the families of these patients.

There are currently no effective therapies for arresting (and, more importantly, reversing) the impairment of central and peripheral nervous system function once an irreversible degenerative cascade begins. Likewise, there is no current therapy for restoration of normal, central and peripheral nervous system function when the induced stress has a less catastrophic or partially reversible effect compared to the dementias.

### SUMMARY OF THE INVENTION

Compositions and therapies for the treatment of neurological disorders are disclosed which compositions are identified by an assay which determines the ability of a test compound to affect neuronal remodeling. Specifically, the assay involves cell cultures which are engineered to affect the expression of different isoforms of apolipoprotein such as apoE3 and/or apoE4 in a manner which results in effects on neuronal remodeling, and neurite outgrowth. Apolipoprotein E3-enriched lipoproteins stimulate outgrowth and microtubule stability whereas apoE4-enriched lipoproteins inhibit outgrowth and disrupt microtubules. Because the inhibition of neuronal remodeling and neurite outgrowth are closely associated with certain diseases of the central nervous system, the assay is useful in screening compounds for potential efficacy in treating such diseases. Compounds which stimulate neural outgrowth and microtubule stability are disclosed as are methods of treating diseases of the central nervous system with such compounds. Differential accumulation of apoE3 and apoE4 is mediated primarily by cell-surface heparin sulfate proteoglycans (HSPG). The retention of both apoE3 and apoE4 is reduced and the differential accumulation of apoE3 and apoE4 is eliminated in (1) cells not expressing any proteoglycan and cells specifically not expressing HSPG and in (2) HSPG-expressing cells treated with heparinase.

Results provided here clearly show that apoliproteins and the differential uptake and/or expressions of different isoforms of these proteins affect nerve cell growth and as such play a significant role in neurological diseases. Further, results shown here demonstrate that proteoglycans in general and specifically heparin sulfate proteoglycans effect differential accumulation of apoE3 and apoE4. Thus, those results allow the production of assays which include cell lines specifically engineered to mimic either hindered or enhanced nerve cell growth thereby making it possible to assay compounds for either their potential as therapeutics or their potential harmful effects on nerve cell growth.

The assay systems and transfected cell lines of the invention can be used not only to screen for potential therapeutic compounds for treating neurological disorders but for determining which compounds would be expected to have an adverse affect on nerve cells and as such should be avoided.

The invention further provides compounds that bind to apoE4 and reduce domain interaction without affecting apoE3. Such compounds render apoE4 more “apoE3-like,” and are therefore useful for treating disorders associated with apoE4, including neurological disorders, neurodegenerative disorders, and disorders caused by hyperlipidemia, e.g., cardiovascular disorders.

The invention further provides methods of treating disorders related to apoE4. In some embodiments, the methods comprise administering a compound that reduces apoE4 domain interaction. Disorders related to apoE4 include neurological disorders and cardiovascular disorders.

An object of the invention is to provide compounds, compositions and methods of using such in the treatment of neurological disease.

Another object of the invention is to provide an assay for testing compounds for their ability to effect neurite outgrowth.

Another object of the invention is to provide an assay for compounds as well as compounds and compositions which affect the differential cellular accumulation of apoE3 and apoE4.

Another object is to provide an assay for compounds as well as compounds and compositions which affect cell-surface HSPG.

Another object is to provide an assay for compounds as well as compounds and compositions which affect the internalization and accumulation of apoE in cells.

A specific object is to provide a cell culture wherein the cells have been genetically engineered with regard to their expression of an apoE protein and to use the cell culture in a screening assay.

An advantage of the invention is that the cell cultures provide a clear indication of the effect of a compound on neurite outgrowth.

Another advantage of the invention is that it can be used to determine which compounds are potentially harmful due to their inhibition of neurite outgrowth and which compounds are potentially therapeutic due to their enhancement of neurite outgrowth.

A feature of the invention is that genes expressing the different isoforms of apoE protein can be individually affected.

The invention also includes methods of identifying compounds that are effective in interfering with the apoE4 domain interaction. These methods are exemplified by the plasma distribution assay comprising the steps of adding a tracer dose of ¹²⁵I-labeled apoE to plasma, separating the various plasma lipoprotein fractions by gel filtration and determining the distribution of ¹²⁵I-label among lipoprotein classes. See, e.g. Dong et al. (1994) J. Biol. Chem. 269:22358-22365.

These and other objects, advantages, and features of the invention will become apparent to those skilled in the art upon reading this disclosure along with the attached figures.

### BRIEF DESCRIPTION OF THE DRAWINGS

FIG. 1 is a schematic representation of the human apoE cDNA constructs used to transfect the Neuro-2a cells. NSE promoter (N), exons of apoE have “E” underneath, the polylinker region has “P” underneath and apoE cDNA has “A” underneath.

FIG. 2 includes 2A, 2B and 2C which are a series of bar graphs depicting the effect of β-VLDL on the number of neurites per cell (A), neurite branching (B), and neurite extension (C) from control Neuro-2a cells and from cells stably transfected to express apoE3 or apoE4. In each case, the solid black bars represent the control, the striped bars represent apoE3 expressing cells and the solid white bars represent apoE4 expressing cells. In all cases the X-axis represents β-VLDL (Tg cholesterol/ml).

FIG. 3 is a graph depicting the effect of β-VLDL on the percentage of cells expressing neurites. Four different fields in each dish were selected, and the percentage of cells displaying neurites was measured. Data are the means of three different experiments performed in duplicate (± S.E.M.). The percentages of cells expressing neurites in the absence of β-VLDL were: control cells, 35±11 (open squares); apoE3-expressing cells, 32±9 (closed circles); apoE4-expressing cells, 25±13 (closed squares). *p<0.025 versus control; **p<0.005 versus control.

FIG. 4 is a bar graph depicting the effect of cerebrospinal fluid (CSF) lipoproteins on neurite extensions from Neuro-2a cells stably transfected to express apoE3 or apoE4. Cells were incubated with β-VLDL or bovine CSF lipoproteins (d<1.21 g/ml). Each data point represents the measurement of 20-40 neurons. The data are reported as the mean ±S.E.M. The solid black bars represent the control. The striped bars represent apoE3 expressing cells. The solid white bars represent apoE4 expressing cells. *p<0.025, **p<0.01, ***p<0.005.

FIG. 5 is a graph of the amount of ¹²⁵I-β-VLDL associated with the particular cells of the invention as graphed over time in hours.

FIG. 6 is a bar graph of the relative fluorescence intensity of the DiI-β-VLDL associated with cells for three different types of cells as labeled.

FIG. 7 is a bar graph of the amount of cholesterol in μg/mg of cell protein for the four different types of cells as labeled.

FIG. 8 is a graph of the relative fluorescence intensity of ApoE over time in hours.

FIG. 9 is a graph of the amount of cell associated ¹²⁵I-ApoE over time for two different types of cells.

FIG. 10 is a graph of the amount of ¹²⁵I-ApoE degraded over time for two different types of cells.

FIG. 11 is a graph of the amount of ¹²⁵I-ApoE which is internalized by two different types of cells over time as measured in hours.

FIG. 12 is a graph of the amount of ¹²⁵I-ApoE degraded over time for two different types of cells as measured in hours.

FIG. 13 is a graph of the amount of ¹²⁵I-ApoE internalized by two different types of cells relative to the concentration of ¹²⁵I-ApoE added to the cell culture.

FIG. 14 is a bar graph of the total amount of ¹²⁵I-ApoE internalized by the two different types of cells tested.

FIG. 15 is a bar graph of the amount of ¹²⁵I-ApoE internalized by human fibroblasts expressing or lacking the LDL receptor.

FIG. 16 is a bar graph of the amount of ¹²⁵I-ApoE internalized by two different types of cells expressing or lacking LRP.

FIG. 17 is a bar graph of the amount of ¹²⁵I-ApoE internalized for the different types of cells as labeled.

FIG. 18 is a bar graph of ¹²⁵I-ApoE associated with the different types of cells as labeled.

FIG. 19 is a bar graph of the amount of ¹²⁵I-ApoE in ng/mg of cell protein for the different types of CHO cells as labeled.

FIG. 20 is a bar graph of the amount of ¹²⁵I-ApoE in Ng/mg of cell protein for the different types of HSPG-deficient CHO cells as labeled.

### DEFINITIONS AND ABBREVIATIONS

Before the present assays and methods are disclosed and described, it is to be understood that this invention is not limited to particular cell lines, reagents, etc., assays or method as such may, of course vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

The publications discussed herein are provided solely for the disclosure prior to the filing date of the present application. Nothing herein is to be construed as an admission that the present invention is not entitled to antedate such publication by virtue of prior invention. Further, the dates of publication provided are subject to change if it is found that the actual date of publication is different from that provided here.

The abbreviations used are:

apoE3, apolipoprotein 3;

apoE4, apolipoprotein 4;

CHO, Chinese hamster ovary;

DiI, 1,1′-dioctadecyl-3,3,3′,3′-tetramethylindocarbocyanine;

DMEM, Dulbecco's modified Eagle's medium;

FBS, fetal bovine serum;

FGF, fibroblast growth factor;

GPI, glycerophophatidylinositol;

HSPG, heparin sulfate proteoglycans;

LDL, low density lipoproteins;

LRP, LDL receptor-related protein;

PBS, phosphate-buffered saline;

SDS-PAGE, sodium dodecyl sulfate-polyacrylamide gel electrophoresis;

TCA, trichloroacetic acid;

VLDL, very low density lipoproteins.

As used herein, an “apoE4-associated disorder” is any disorder that is caused by the presence of apoE4 in a cell, in the serum, in the interstitial fluid, in the cerebrospinal fluid, or in any other bodily fluid of an individual; any physiological process or metabolic event that is influenced by apoE4 domain interaction; any disorder that is characterized by the presence of apoE4; a symptom of a disorder that is caused by the presence of apoE4 in a cell or in a bodily fluid; a phenomenon associated with a disorder caused by the presence in a cell or in a bodily fluid of apoE4; and the sequelae of any disorder that is caused by the presence of apoE4. ApoE4-associated disorders include apoE4-associated neurological disorders and disorders related to high serum lipid levels. ApoE4-associated neurological disorders include, but are not limited to, sporadic Alzheimer's disease; familial Alzheimer's disease; poor outcome following a stroke; poor outcome following traumatic head injury; and cerebral ischemia. Phenomena associated with apoE4-associated neurological disorders include, but are not limited to, neurofibrillary tangles; amyloid deposits; memory loss; and a reduction in cognitive function. ApoE4-related disorders associated with high serum lipid levels include, but are not limited to, atherosclerosis, and coronary artery disease. Phenomena associated with such apoE4-associated disorders include high serum cholesterol levels.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” and “patient,” used interchangeably herein, refer to a mammal, including, but not limited to, murines, simians, humans, mammalian farm animals, mammalian sport animals, and mammalian pets.

### DETAILED DESCRIPTION OF PREFERRED EMBODIMENTS

Agents that Reduce ApoE4 Domain Interaction

The invention provides agents affecting apoE4 domain interaction, and compositions comprising such agents. By reducing apoE4 domain interaction, apoE4 is rendered more “apoE3-like,” and the undesirable effects of apoE4 are reduced. Agents that reduce apoE4 domain interactions are useful in treating apoE4-associated neurological disorders. Agents that reduce apoE4 domain interaction are also useful in treating apoE4-associated disorders related to high serum lipid levels, e.g., cardiovascular disorders.

Agents that reduce apoE4 domain interaction include agents that inhibit formation of a salt bridge between arg-61 and glu-255. Agents of interest are those that reduce apoE4 domain interaction by at least about 10%, at least about 20%, at least about 25%, at least about 30%, at least about 40%, at least about 50%, at least about 60%, at least about 70%, at least about 80%, at least about 90%, or at least about 95% or more, up to 100%, compared to apoE4 domain interaction in the absence of the agent.

Agents of interest are those that affect apoE4 domain interaction without substantially affecting apoE3 structure, i.e., the effect on apoE4 domain interaction is specific to apoE4. Whether an agent specifically reduces apoE4 domain interaction can be determined using an assay such as the emulsion binding assay described in Example 7.

In some embodiments, an agent that reduces apoE4 domain interaction renders the apoE4 molecule more “apoE3-like,” e.g., the apoE4 molecule has apoE3 activity. Thus, in some embodiments, the invention provides methods for converting apoE4 activity to apoE3 activity, comprising contacting an apoE4 molecule with an agent that reduces apoE4 domain interaction. Characteristics of “apoE4 activity” and “apoE3 activity” include, but are not limited to, binding preference of the apolipoprotein for a particular class of lipoprotein; binding to tau protein in vitro and/or in vivo; and binding to Aβ protein. In some embodiments, an agent that reduces apoE4 domain interaction converts apoE4 activity to apoE3 activity such that the apoE4, when contacted with the agent that reduces apoE4 domain interaction, reduces a characteristic of apoE4 by at least about 10%, at least about 20%, at least about 30%, at least about 40%, at least about 50%, at least about 60%, at least about 70%, at least about 80%, at least about 90%, or more, when compared with the characteristic of apoE4 in the absence of the agent.

ApoE4 has a binding preference for VLDL, while apoE3 has a binding preference for HDL. Typically, when plasma lipoproteins are allowed to bind to labeled apoE4 and apoE3, the bound proteins fractionated, and the amount of apoE4 and apoE3 in each fraction measured, the amount of apoE4 in the VLDL, IDL/LDL, and HDL fractions is about 35%, about 23%, about 42%, respectively, while the amount of apoE3 in each of these fractions is about 20%, about 20%, about 60%, respectively. Thus, in some embodiments, an agent that reduces apoE4 domain interaction causes apoE4 to have a binding preference for HDL. Whether apoE4, when contacted with an agent that reduces apoE4 domain interaction, has a binding preference for HDL over VLDL can be determined using any known assay. As one non-limiting example, an assay as described in Dong et al. (1994) J. Biol. Chem. 269:22358-22365. For example, samples comprising detectably labeled apoE4 and apoE3 (e.g., labeled with ¹²⁵I), are mixed with plasma at about 37° C. for about 2 hours, after which time the samples are fractionated into various lipoprotein classes (e.g., by chromatography), and the amount of label in each fraction is determined.

ApoE3 interacts with tau in vitro, while apoE4 does not. In some embodiments, an agent that reduces apoE4 domain interaction causes apoE4 to bind tau in vitro and/or in vivo. Whether a protein binds tau in vitro, e.g., in the presence of an agent that reduces apoE4 domain interaction, can be determined using standard assays for measuring or detecting protein-protein interaction. A non-limiting example of an assay is provided in Strittmatter et al. (1994) Exp. Neurol. 125:163-171.

In many embodiments, agents that reduce apoE4 domain interaction are small organic molecules, generally in the size range of from about 50 daltons to about 2500 daltons, from about 100 daltons to about 2000 daltons, from about 200 daltons to about 1500 daltons, from about 300 daltons to about 1250 daltons, or from about 500 daltons to about 1000 daltons.

The terms “agent”, “substance,” and “compound” are used interchangeably herein. Candidate agents encompass numerous chemical classes, typically synthetic, semi-synthetic, or naturally-occurring inorganic or organic molecules. Candidate agents may be small organic compounds having a molecular weight of more than about 50 daltons and less than about 2,500 daltons. Candidate agents may comprise functional groups necessary for structural interaction with proteins, e.g., van der Waals interactions, hydrogen bonding, and the like, and may include at an amine, a sulfoalkyl, a carbonyl, a hydroxyl, or a carboxyl group, and may contain at least two of the aforementioned functional chemical groups. The candidate agents may comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries.

Pharmacological agents may be subjected to directed or random and/or directed chemical modifications, such as acylation, alkylation, esterification, amidification, etc. to produce structural analogs. Such structural analogs include those that increase bioavailability, and/or reduced cytotoxicity. Those skilled in the art can readily envision and generate a wide variety of structural analogs, and test them for desired properties such as increased bioavailability and/or reduced cytotoxicity and/or ability to cross the blood-brain barriers.

In some embodiments, a compound that reduces apoE4 domain interaction is a member of a family of structurally related compounds, including, but not limited to, a blocked amino acid; a disulfonate; a dye; a monosulfate; and a monosulfoalkyl compound.

In particular embodiments, a compound that reduces apoE4 domain interaction is selected from the group consisting of Z-D-Tyr(BZL)—OH, azocarnine G, glycine cresol red, erythrosin B, 5-chloro-2-(4-chloro-2-(3,4-dichloro) phenylureido, RCL S19,214-7, 3-butyl-1-ethyl-5-2-(3-sulfobutyl-benzo (1,3) oxazo, or a structural analog of any of the foregoing.

In many embodiments, agents that reduce apoE4 domain interaction reduce apoE4-mediated inhibition of neurite outgrowth. Whether a compound reduces apoE4-mediated inhibition of neurite outgrowth can be determined using a neurite outgrowth assay as described herein. In general, an agent that reduces apoE4 domain interaction reduces apoE4-mediated inhibition of neurite outgrowth by at least about 10%, at least about 20%, at least about 30%, at least about 40%, at least about 50%, or more, when compared to the inhibition of neurite outgrowth in the presence of apoE4 and the absence of the agent.

Many methods are available to identify agents that reduce apoE4 domain interaction. As one non-limiting example, one can use computer modeling to identify compounds that bind to the N-terminal domain of apoE4. Computer modeling programs are known in the art and include, but are not limited to, the DOCK program, as described in Example 7.

Compounds that bind to the N-terminal domain of apoE4 based on computer modeling may be further evaluated, e.g., by functional assays. Functional assays, include, but are not limited to, an emulsion binding assay (as described in Example 7), assays measuring binding to an LDL receptor, assays measuring binding to LRP, assays measuring binding to HSPG, and neurite outgrowth assays.

Also of interest is an agent that reduces apoE4 domain interaction that also reduces formation of neurofibrillary tangles in an individual. In these embodiments, an agent that reduce apoE4 domain interaction and that reduces formation of neurofibrillary tangles reduces formation of neurofibrillary tangles by at least about 10%, at least about 20%, at least about 30%, at least about 40%, at least about 50%, at least about 60%, at least about 70%, at least about 80%, or at least about 90%, when compared to formation of neurofibrillary tangles in the absence of the agent. Whether neurofibrillary tangle formation is reduced can be determined using, e.g., an experimental animal model of Alzheimer's disease, wherein the animal synthesizes human apoE4 and, as a result, produces neurofibrillary tangles. See, e.g. U.S. Pat. No. 6,046,381.

Agents that reduce apoE4 domain interaction to the desired extent may also be assessed for cellular availability, cytotoxicity, biocompatibility, ability to cross the blood-brain barrier, etc., using standard assays.

The invention further provides compositions comprising an agent that reduces apoE4 domain interaction. These compositions may include a buffer, which is selected according to the desired use of the agent, and may also include other substances appropriate to the intended use. Those skilled in the art can readily select an appropriate buffer, a wide variety of which are known in the art, suitable for an intended use. In some instances, the composition can comprise a pharmaceutically acceptable excipient, a variety of which are known in the art and need not be discussed in detail herein. Pharmaceutically acceptable excipients have been amply described in a variety of publications, including, for example, A. Gennaro (1995) “Remington: The Science and Practice of Pharmacy”, 19th edition, Lippincott, Williams, & Wilkins.

FORMULATIONS, DOSAGES, AND ROUTES OF ADMINISTRATION

The invention provides formulations, including pharmaceutical formulations, comprising an agent that reduces apoE4 domain interaction. In general, a formulation comprises an effective amount of an agent that reduces apoE4 domain interaction. An “effective amount” means a dosage sufficient to produce a desired result, e.g., reduction in apoE4 domain interaction, an increase in neurite outgrowth, a reduction in serum lipid levels, a reduced risk of heart disease, etc. Generally, the desired result is at least a reduction in apoE4 domain interaction as compared to a control. An agent that reduces apoE4 domain interaction may delivered in such a manner as to avoid the blood-brain barrier, as described in more detail below. An agent that reduces apoE4 domain interaction may be formulated and/or modified to enable the agent to cross the blood-brain barrier, as described in more detail below.

Formulations

In the subject methods, the active agent(s) may be administered to the host using any convenient means capable of resulting in the desired reduction in apoE4 domain interaction. Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

Other modes of administration will also find use with the subject invention. For instance, an agent of the invention can be formulated in suppositories and, in some cases, aerosol and intranasal compositions. For suppositories, the vehicle composition will include traditional binders and carriers such as, polyalkylene glycols, or triglycerides. Such suppositories may be formed from mixtures containing the active ingredient in the range of about 0.5% to about 10% (w/w), preferably about 1% to about 2%.

Intranasal formulations will usually include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa.

An agent of the invention can be administered as injectables. Typically, injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, eg., Remington's Pharmaceutical Sciences, Mack Publishing Company, Easton, Pa., 17th edition, 1985. The composition or formulation to be administered will, in any event, contain a quantity of the agent adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Dosages

Although the dosage used will vary depending on the clinical goals to be achieved, a suitable dosage range is one which provides up to about 1 μg to about 1,000 μg or about 10,000 μg of an agent that reduces apoE4 domain interaction and can be administered in a single dose. Alternatively, a target dosage of an agent that reduces apoE4 domain interaction can be considered to be about in the range of about 0.1-1000 μM, about 0.5-500 μM, about 1-100 μM, or about 5-50 μM in a sample of host blood drawn within the first 24-48 hours after administration of the agent.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

Routes of Administration

An agent that reduces apoE4 domain interaction is administered to an individual using any available method and route suitable for drug delivery, including in vivo and ex vivo methods, as well as systemic and localized routes of administration.

Conventional and pharmaceutically acceptable routes of administration include intranasal, intramuscular, intratracheal, intratumoral, subcutaneous, intradermal, topical application, intravenous, rectal, nasal, oral and other parenteral routes of administration. Routes of administration may be combined, if desired, or adjusted depending upon the agent and/or the desired effect. The composition can be administered in a single dose or in multiple doses.

The agent can be administered to a host using any available conventional methods and routes suitable for delivery of conventional drugs, including systemic or localized routes. In general, routes of administration contemplated by the invention include, but are not necessarily limited to, enteral, parenteral, or inhalational routes.

Parenteral routes of administration other than inhalation administration include, but are not necessarily limited to, topical, transdermal, subcutaneous, intramuscular, intraorbital, intracapsular, intraspinal, intrasternal, and intravenous routes, i.e., any route of administration other than through the alimentary canal. Parenteral administration can be carried to effect systemic or local delivery of the agent. Where systemic delivery is desired, administration typically involves invasive or systemically absorbed topical or mucosal administration of pharmaceutical preparations.

The agent can also be delivered to the subject by enteral administration. Enteral routes of administration include, but are not necessarily limited to, oral and rectal (e.g., using a suppository) delivery.

Methods of administration of the agent through the skin or mucosa include, but are not necessarily limited to, topical application of a suitable pharmaceutical preparation, transdermal transmission, injection and epidermal administration. For transdermal transmission, absorption promoters or iontophoresis are suitable methods. lontophoretic transmission may be accomplished using commercially available “patches” which deliver their product continuously via electric pulses through unbroken skin for periods of several days or more.

By treatment is meant at least an amelioration of the symptoms associated with the pathological condition afflicting the host, where amelioration is used in a broad sense to refer to at least a reduction in the magnitude of a parameter, e.g. symptom, associated with the pathological condition being treated, such as an apoE4-associated neurological disorder and pain associated therewith. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

A variety of hosts (wherein the term “host” is used interchangeably herein with the terms “subject” and “patient”) are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

Kits with unit doses of the active agent, e.g. in oral or injectable doses, are provided. In such kits, in addition to the containers containing the unit doses will be an informational package insert describing the use and attendant benefits of the drugs in treating pathological condition of interest. Preferred compounds and unit doses are those described herein above.

Methods of Treating apoE4-associated Neurological Disorders

The invention further provides methods of treating apoE4 neurological disorders. In some embodiments, the invention provides methods for reducing apoE4 domain interaction in a host cell that synthesizes apoE4, comprising administering an effective amount of an agent that reduces apoE4 domain interaction to an individual in need thereof. In other embodiments, the invention provides methods for reducing apoE4 domain interaction in apoE4 that is extracellular, e.g., in the serum, cerebrospinal fluid, or in the interstitial fluid. In some embodiments, an agent that reduces apoE4 domain interaction is one that is effective in increasing neurite outgrowth. In other embodiments, an agent that reduces apoE4 domain interaction is one that results in improved outcome following stroke. In some embodiments, an agent that reduces apoE4 domain interaction is one that is effective in increasing neurite outgrowth. In other embodiments, an agent that reduces apoE4 domain interaction is one that results in improved outcome following traumatic head injury. In other embodiments, an agent that reduces apoE4 domain interaction is one that reduces the risk of developing Alzheimer's disease. In other embodiments, an agent that reduces apoE4 domain interaction is one that reduces a symptom or phenomenon associated with Alzheimer's disease. In some of these embodiments, an agent that reduces apoE4 domain interaction is one that reduces formation of neurofibrillary tangles. In other embodiments, an agent that reduces apoE4 domain interaction is one that, when administered to an individual, results in reduced amyloid deposits in the brain of the individual.

In some embodiments, an agent that reduces apoE4 domain interaction reduces a symptom associated with AD, such as formation of neurofibrillary tangles or Aβ deposits, by at least about 10%, at least about 20%, at least about 30%, at least about 50%, at least about 60%, at least about 70%, at least about 80%, at least about 90% or more. In other embodiments, an agent that reduces apoE4 domain interaction improves a parameter that is in decline in individuals with AD, such as memory or cognitive function, by at least about 10%, at least about 20%, at least about 30%, at least about 50%, at least about 60%, at least about 70%, at least about 80%, at least about 90% or more, such that the decline in one of these parameters is at least slowed.

Neuronal cells may produce apoE4 themselves. Alternatively, or in addition, neuronal cells may take up apoE4 from their environment, e.g., apoE4 produced by supporting cells such as astrocytes and glial cells and secreted into the interstitial fluid.

In some embodiments, the methods of the invention are effective in reducing apoE4 domain interaction in neuronal cells that produce apoE4 and/or that take up apoE4 from their environment, i.e., neuronal cells in which detectable amounts of apoE4 are found. Neuronal cells amenable to treatment using the methods of the invention include those that produce or take up from about 1 ng to about 1000 ng (or more), from about 5 ng to about 500 ng, from about 10 ng to about 100 ng, apoE4 per mg total cell protein in a 48-hour period.

In other embodiments, the invention provides methods for inhibiting formation of neurofibrillary tangles in an individual, comprising administering an effective amount of an agent that reduces apoE4 domain interaction to the individual. Whether formation of neurofibrillary tangles is inhibited can be determined, e.g., in experimental animal models of Alzheimer's disease (AD). Experimental animal models of AD have been described in the art; any known animal model of AD can be used to determine whether an agent of the invention inhibits formation of neurofibrillary tangles. See, e.g., U.S. Pat. No.6,046,381. Such animal models can also be used to determine whether other phenomena, such as amyloid deposition, and cognitive abilities, are affected by an agent that reduces apoE4 domain interaction. Whether an agent that reduces apoE4 domain interaction reduces formation of neurofibrillary tangles and/or Aβ deposits can also be determined in humans using any known method, including, but not limited to, immunohistochemical staining of brain biopsy samples.

In other embodiments, the invention provides methods for treating AD, comprising administering to an individual an effective amount of an agent that reduces apoE4 domain interaction. Individuals known to be at risk of developing AD are amenable to treatment using the methods of the invention. Thus, an agent that reduces apoE4 domain interaction is suitable for use prophylactically in patients who are heterozygous or homozygous for apoE4 but do not show overt symptoms of Alzheimer's disease or other neurodegenerative disorders. The methods are also useful to treat an individual who already displays symptoms of AD, where the method treats AD by reducing advancement of the disease, or reduces severity of a symptom associated with AD. Whether advancement of AD is reduced or severity of an AD-related symptom is reduced can be determined by assessing any symptom or parameter associated with AD, including, but not limited to, cognitive function, and memory. Such determinations are well within the ability of those skilled in the art using standard methods known in the art.

In some embodiments, an agent that reduces apoE4 domain interaction is one that, when administered to an individual in need thereof, such as a stroke patient or an individual who has undergone traumatic head injury, improves the clinical outcome for that individual. Whether an agent that reduces apoE4 domain interaction results in improved outcome following stroke or traumatic head injury when the agent is administered to an individual who has suffered a stroke or traumatic head injury can be determined using any available animal model of stroke and traumatic head injury. Rodent models of neuronal damage, for example neuronal damage caused by cerebral ischemia, may be examined to determine the effect on an agent that reduces apoE4 domain interaction on the extent of neuronal damage caused by traumatic events as well as their role in neuronal remodeling, repair and recovery from such insults. Rodent models of cerebral ischemia, both global ischemia and focal ischemia, are useful for studying mechanisms controlling the occurrence of cerebral ischemia and potential therapeutic strategies for treatment of injury caused by ischemic events. Animal models of global ischemia, which is usually transient, have widely affected brain areas but typically give rise to neuronal alterations in selectively vulnerable brain regions. Examples of such models include, but are not limited to, the two vessel occlusion model of forebrain ischemia, the four vessel occlusion model of forebrain ischemia, and ischemia models involving elevated cerebrospinal fluid pressure. See, e.g., Ginsberg and Busto, Stroke, 20:1627-1642 (1989).

Methods for Treating ApoE4-Related Disorders Associated With Hyperlipidemia

The invention further provides methods for treating apoE4-related disorders that are associated with elevated serum lipid levels. The methods generally comprise administering to an individual an effective amount of an agent that reduces apoE4 domain interaction.

In some embodiments, the invention provides methods for reducing serum cholesterol levels, comprising administering an agent that reduces apoE4 domain interaction. In these embodiments, an agent that reduces apoE4 domain interaction reduces serum cholesterol levels in an individual when administered to the individual by at least about 10%, at least about 20%, at least about 30%, at least about 40%, or at least about 50%, compared to a serum cholesterol in an individual not administered with the agent. In general, an effective amount of an agent that reduces apoE4 domain interaction is effective at least in reducing a serum cholesterol level such that it is in a normal range. A normal range of serum cholesterol will vary, depending upon the sex and age of the individual, as well as other factors. For adult humans, a normal range of serum cholesterol is from about 200 to about 240 mg/dL. An “elevated serum cholesterol level” is similary dependent upon age and sex of the individual. Thus, e.g., an adult human having a serum cholesterol level of over 240 mg/dL is considered to have an elevated serum cholesterol level. In some embodiments, an effective amount of an agent that reduces apoE4 domain interaction is one that is effective in reducing serum cholesterol levels to below 240 mg/dL.

In other embodiments, the invention provides methods of reducing the risk that an individual will develop coronary artery disease (CAD) or atherosclerosis, comprising administering to the individual an effective amount of an agent that reduces apoE4 domain interaction. In these embodiments, an agent that reduces apoE4 domain interaction reduces the risk of developing CAD or atherosclerosis by at least about 10%, at least about 20%, at least about 30%, at least about 40%, or at least about 50% or more, when compared with the risk associated with an individual not treated with the agent.

Individuals who are amenable to treatment with the methods of the invention include those who are known to be at risk for developing CAD because these individuals express apoE4; individuals who express apoE4 and have elevated serum cholesterol levels; and individuals who express apoE4 and have had one or more cardiac events.

Assays to Detect Compounds Affecting Neuronal Cell Growth

Differential expression of different isoforms of apolipoprotein E affects neuronal cell growth. In some embodiments, assays of the invention utilize differential expression of different isoforms of apolipoprotein E in order to determine compounds which affect neuronal cell growth. In other embodiments, assays described herein identify compounds that reduce apoE4 domain interaction. Compounds identified via an assay of the invention are formulated into compositions which are useful in the treatment of neurological diseases—particularly such diseases where abnormal differential expression of isoforms of apolipoproteins is present. Details regarding theories behind the invention as well as specific examples of the invention are provided below. However, the invention is not limited by such theories or examples.

In neurons, the cytoskeleton functions in neurite extension and retraction. Therefore, the studies described herein and by others (Handelmann (1992); and Nathan et al. (1994) Science 264:850-852), have focused on the isoform-specific effects of apoE3 and apoE4 on neurite extension and branching. Different isoforms of apoE modulate the intracellular cytoskeletal apparatus and alter neurite extension and branching. Understanding how the various apoE isoforms alter the cytoskeleton provides information on (1) the process of neurofibrillary tangle formation and (2) control of apoE-induced remodeling of synaptic connections later in life. Compounds which stimulate neurite extension in vivo are likely to promote nerve regeneration or the formation of synaptic connections during neuronal remodeling in both the central and peripheral nervous system.

We have developed specific assays for screening compounds for their effect on neuronal growth. Further, the assay makes it possible to screen for compounds which affect cell-surface HSPG and thereby effect differential cellular accumulation of apoE3 and apoE4.A comparison of the effects of human apoE3 versus human apoE4 showed pronounced differential isoform-specific effects on neurite outgrowth. Compared to a control, human apoE3 plus β-VLDL resulted in an increase in neurite extension, while apoE4 plus β-VLDL resulted in a marked decrease in both neurite branching and extension. Results presented by Nathan et al. (1995) show that dorsal root ganglion neurons incubated with apoE4 plus β-VLDL displayed very short, stunted neurites. This was not a toxic effect of apoE4 since replacement of the apoE4-containing media with fresh apoE4-lacking media restored the ability of the neurons to produce neuritic extensions. Furthermore, the apoE3- and apoE4-specific effects were blocked by (1) an antibody against the receptor binding domain of apoE or (2) reductive methylation of critical lysine residues, indicating that this effect of apoE is receptor-mediated, or HSPG-mediated.

Neuro-2a cells from the central nervous system were used to compare the effects of apoE on the peripheral nervous system neurons described above with the effect on cortical neurons. Cells of both types respond similarly to apoE. When combined with a source of lipid, apoE3 stimulated neurite extension, whereas apoE4 inhibited neurite extension. Nathan et al. (1994) Soc. Neurosci. 20 (Part 2):1033 (Abstr.); and Nathan et al. (1995). Addition of free apoE3 or apoE4 without β-VLDL had no effect on neurite outgrowth. These results indicate that the effect of apoE on neurons requires the lipoprotein receptor-mediated uptake of apoE or a combination of apoE and lipid. Free of lipid, apoE does not bind to either the LDL receptor or the LRP. In contrast, in another study, using a different neuronal cell line, Holtzman et al. demonstrated that apoE3 with β-VLDL stimulated nerve growth factor-induced neurite outgrowth, whereas apoE4 had no effect. Holtzman et al. (1995) Soc. Neurosci. 21 (abstr): 1009, 400.10.

To determine whether lower levels of endogenously produced apoE would have an effect on neurite outgrowth from Neuro-2a cells, in the examples provided below, the neuronal cells were transfected with human apoE cDNA constructs encoding apoE3 or apoE4. Clones of the transfected cells secreting equal amounts of apoE3 or apoE4 (˜50-60 ng of apoE/mg of cell protein/48 hours) were selected for comparison. The apoE3- and apoE4-secreting cells grown in serum-free control medium displayed a similar degree of limited neurite extension. However, when a source of lipid (β-VLDL) was added to the medium, the cells had a markedly different growth pattern. The apoE3-secreting cells showed greater neurite extension than did the apoE4-secreting cells. Thus, even very low levels of endogenously produced apoE along with a source of lipid revealed the differential effects of apoE3 versus apoE4. Lipid emulsions of various compositions, as well as cerebrospinal fluid lipoproteins can be substituted for the β-VLDL and appear to serve as a source of lipid for the cells or as a vehicle for transporting the apoE into a specific intracellular pathway. The examples presented herein show that the apoE effect on neurite outgrowth is mediated through the LRP, or a similar apoE-binding receptor, and that blocking or effectively preventing this interaction inhibits the apoE4 induced inhibition of neurite outgrowth.

Thus, the invention relates to assaying compounds for their ability to reduce the apoE4-induced inhibition of neuron remodeling by inhibiting the interaction of apoE4 and an apoE-binding receptor, e.g., the LRP. Compounds found via the assay might alter the function of apoE4 by changing the domain interaction to interfere with the inhibition of apoE4 in neuron remodeling. Any agent that blocks the interaction of arginine-6 1 with glutamic acid-255 in apoE4 could be screened for in the assay. Blocking domain interaction in apoE4 converts apoE4 to an “apoE3-like” molecule, thereby blunting the undesirable effects of apoE4 on neurite extension. This may also have the effect of switching the apoE4 binding preference from VLDL to HDL.

Assays can screen for compounds with any effect on neurite growth, but the compounds screened for preferably reduce apoE4 inhibition of neurite outgrowth by at least about 10%, preferably at least about 50% and most preferably, at least about 90%. The effect on neurite outgrowth can be measured, for instance, by the methods described herein.

Assays of the invention can be used to screen for compounds which prevent apoE4 from interacting effectively with neuronal LRP or other apoE-binding receptors. This prevention can be directed at either the HSPG and/or the LRP interactions or by modifying its function to be more apoE3-like and can directly or indirectly block binding or otherwise prevent the signal transduction induced by apoE4 binding. Thus, assays screen for compounds which prevent inhibition of neurite outgrowth by any of these routes. Thus, the invention comprises whole proteins, any functional portion thereof, analog or homologue which prevent effective interaction of apoE4 and HSPG or LRP, or other apoE-binding receptors. For instance, changes in the amino acid sequences of the RAP or lactoferrin and other known ligands of the LRP, or other apoE-binding receptors, that do not substantially affect their ability to effectively block the interaction of apoE4 and the LRP are compounds to be screened for.

The invention also encompasses methods for detecting therapeutic agents that reduce the interaction of apoE4 and the LRP and other members of the LDL receptor family. The methods include in vitro ligand blotting techniques. This can be performed following the separation of cell membrane proteins (which contain the LRP) or the LRP partially purified from membrane proteins for instance by nonreducing sodium dodecylsulfate-polyacrylamide gel electrophoresis and transfer to a nitrocellulose membrane. Methods of partial purification of the LRP are described, for instance, by Schneider et al. (1985) Methods Enzymol. 109:405-417. The membrane is then incubated with apoE and a lipoprotein (e.g. β-VLDL) which is labeled, for instance by biotinylation. Binding of the apoE-β-VLDL complex to the membrane is then visualized using reagents that detect the label. Agents to be tested for their ability to block the interaction are added to the nitrocellulose together with apoE and β-VLDL to determine if the interaction is blocked.

### NEUROLOGICAL DISEASES

Compounds found via an assay described herein are formulated to provide therapeutics for patients suffering from a wide range of disorders. For instance, patients suffering from neurodegeneration or hypoxia may be treated. Neurodegeneration may result from a number of causes, including, but not limited to, Alzheimer's disease, trauma, viral infections, genetic enzyme deficiencies, age-related cognitive decline, and prion diseases. Viruses which may cause neurodegeneration include, but are not limited to, human immunodeficiency virus (HIV) and Epstein-Barr virus. Genetic enzyme deficiencies which may cause neurodegeneration include, but are not limited to, deficiency in β-N-acetylhexosaminidase which causes Tay-Sachs disease. Age-related cognitive decline is described, for instance, in Diagnostic and Statistical Manual of Mental Disorders, Fourth ed., Washington D.C. American Psychiatric Association (1994). Prion diseases include, but are not limited to, Kuru and Creutzfeldt-Jacob disease. Hypoxia is generally the result of stroke or is temporary and associated for instance with drowning, airway obstructions or carbon monoxide poisoning.

Neuron remodeling is also important in otherwise healthy patients. Therefore, compounds identified by the assay may be suitable for use prophylactically in patients who are heterozygous or homozygous for apoE4 but do not show overt symptoms of Alzheimer's disease or other neurodegenerative disorders.

The neurite outgrowth assay of the invention has been used to identify potential therapeutics including glycoprotein such as RAP, heparinases, and lactoferrin all of which reduce or abolish apoE4-induced inhibition of neurite outgrowth. Assays of the present invention can identify compounds that bind specifically to apoE4 and prevent its domain interaction, e.g., small molecules and antibodies. Agents that disrupt the domain interaction can be selected from a wide variety of molecules, including, but not limited to, small molecules, glycoproteins, peptides and antibodies which are designed to bind to arginine-61 or glutamic acid-255 of apoE4. Specific assays for screening for agents that disrupt this domain interaction is described in Example 3 and Example 7, below. Assays of the invention include those that determine whether apoE4 exhibits apoE3 activity.

Heparinases or other modifiers of HSPG are effective in vitro in ameliorating the effects of apoE4 on neuron remodeling. However, their pleiotropic effects render them unsuitable for human therapy. Assays of the invention can be used to identify potentially effective therapeutic agents such as HSPG analogs which bind to apoE4 to prevent its binding to neurons but do not exert substantial pleiotropic effects.

The RAP is a glycoprotein with an apparent molecular mass of 39-kD in humans. The RAP specifically associates with gp330 and the LRP, both of which are members of the LDL receptor gene family. Various RAPs and homologs thereof have been described and their functional domains have been mapped. For review see, Orlando et al. (1994) Proc. Natl. Acad. Sci. USA 91:3161-3165; and Warshawsky et al. (1995) Biochem. 34:3404-3415. The RAP, and portions thereof, are known to block the binding of the LRP to its ligand t-PA and 1₂-macroglobulin-protease complexes. Warshawsky et al. (1994) Ann. N.Y. Acad. Sci. pp. 514-517.

### LACTOFERRIN

Lactoferrin has been shown to bind to the LRP, gp330, and HSPG. Willnow et al. (1994) J. Biol. Chem. 267:26172-26180;, Mahley et al. (1994) Ann. N.Y. Acad. Sci. USA 10 737:39-52; and Ji et al. (1994a) Arterioscler. Thromb. 14:2025-2032. Lactoferrin appears to be cleared from the bloodstream by binding with LRP. Meilinger et al. (1995). Lactoferrin blocks binding of ligands to both the LRP and HSPG and blocks the HSPG-LRP pathway. This apparently occurs through the interaction of a region of concentrated positive charge on the lactoferrin with negatively-charged groups on the HSPG and negatively-charged amino acids in the ligand binding domain of the LRP.

### ANTIBODIES

Antibodies specific for apoE block the apoE4 induced inhibition of neuron remodeling. Assays of the invention can be used to screen antibodies to either apoE4 or the LRP to determine the potential utility therapeutically. The assay can screen antibodies to find those that inhibit the neuron remodeling inhibitory effect of apoE4 whether by inhibiting binding to the LRP or by altering the function of apoE4 to become more apoE3-like. Preferred antibodies are monoclonal and specific for the apoE4 isoform and not apoE3 or apoE2. The term “antibody” also includes functional portions and equivalents thereof. For instance, antibodies include any monospecific compound comprised of a sufficient portion of the light chain variable region to effect binding to the epitope to which the whole antibody has binding specificity. The fragments may include the variable region of at least one heavy or light chain immunoglobulin peptide, and include, but are not limited to, Fab fragments, Fab2 fragments, and Fv fragments. In addition, the monospecific domains of antibodies can be produced by recombinant engineering. Such recombinant molecules include, but are not limited to, fragments produced in bacteria, and murine antibodies in which the majority of the murine constant regions have been replaced with human antibody constant regions.

### DELIVERY OF THERAPEUTIC AGENTS

After an assay of the invention has shown that a compound has certain characteristics as a potential therapeutic it is within the skill of one in the art to determine whether the compound has in vivo therapeutic utility. It is also within the skill of one in the art to formulate suitable dosage formats for delivery of the therapeutic agents. When the site of delivery is the brain, the therapeutic agent must be capable of being delivered to the brain.

The blood-brain barrier limits the uptake of many therapeutic agents into the brain and spinal cord from the general circulation. Molecules which cross the blood-brain barrier use two main mechanisms: free diffusion; and facilitated transport. Because of the presence of the blood-brain barrier, attaining beneficial concentrations of a given therapeutic agent in the CNS may require the use of drug delivery strategies. Delivery of therapeutic agents to the CNS can be achieved by several methods.

One method relies on neurosurgical techniques. In the case of gravely ill patients such as accident victims or those suffering from various forms of dementia, surgical intervention is warranted despite its attendant risks. For instance, therapeutic agents can be delivered by direct physical introduction into the CNS, such as intraventricular or intrathecal injection of drugs. Intraventricular injection may be facilitated by an intraventricular catheter, for example, attached to a reservoir, such as an Ommaya reservoir. Methods of introduction may also be provided by rechargeable or biodegradable devices. Another approach is the disruption of the blood-brain barrier by substances which increase the permeability of the blood-brain barrier. Examples include intra-arterial infusion of poorly diffusible agents such as mannitol, pharmaceuticals which increase cerebrovascular permeability such as etoposide, or vasoactive agents such as leukotrienes. Neuwelt and Rappoport (1984) Fed. Proc. 43:214-219; Baba et al. (1991) J. Cereb. Blood Flow Metab. 11:638-643; and Gennuso et al. (1993) Cancer Invest. 11:638-643.

Further, it may be desirable to administer the pharmaceutical agents locally to the area in need of treatment; this may be achieved by, for example, local infusion during surgery, by injection, by means of a catheter, or by means of an implant, said implant being of a porous, non-porous, or gelatinous material, including membranes, such as silastic membranes, or fibers.

Therapeutic compounds can also be delivered by using pharmacological techniques including chemical modification or screening for an analog which will cross the blood-brain barrier. The compound may be modified to increase the hydrophobicity of the molecule, decrease net charge or molecular weight of the molecule, or modify the molecule, so that it will resemble one normally transported across the blood-brain barrier. Levin (1980) J. Med. Chem. 23:682-684; Pardridge (1991) in: Peptide Drug Delivery to the Brain; and Kostis et al. (1994) J. Clin. Pharmacol. 34:989-996.

Encapsulation of the drug in a hydrophobic environment such as liposomes is also effective in delivering drugs to the CNS. For example WO 91/04014 describes a liposomal delivery system in which the drug is encapsulated within liposomes to which molecules have been added that are normally transported across the blood-brain barrier.

Another method of formulating the drug to pass through the blood-brain barrier is to encapsulate the drug in a cyclodextrin. Any suitable cyclodextrin which passes through the blood-brain barrier may be employed, including, but not limited to, J-cyclodextrin, K-cyclodextrin and derivatives thereof. See generally, U.S. Pat. Nos. 5,017,566, 5,002,935 and 4,983,586. Such compositions may also include a glycerol derivative as described by U.S. Pat. No. 5,153,179.

Delivery may also be obtained by conjugation of a therapeutic agent to a transportable agent to yield a new chimeric transportable therapeutic agent. For example, vasoactive intestinal peptide analog (VIPa) exerted its vasoactive effects only after conjugation to a monoclonal antibody (Mab) to the specific carrier molecule transferrin receptor, which facilitated the uptake of the VIPa-Mab conjugate through the blood-brain barrier. Pardridge (1991); and Bickel et al. (1993) Proc. Natl. Acad Sci. USA 90:2618-2622. Several other specific transport systems have been identified, these include, but are not limited to, those for transferring insulin, or insulin-like growth factors I and II. Other suitable, non-specific carriers include, but are not limited to, pyridinium, fatty acids, inositol, cholesterol, and glucose derivatives. Certain prodrugs have been described whereby, upon entering the central nervous system, the drug is cleaved from the carrier to release the active drug. U.S. Pat. No. 5,017,566.

### EXAMPLES

The following examples are put forth so as to provide those of ordinary skill in the art with a complete disclosure and description of how to carry out the invention and are not intended to limit the scope of what the inventors regard as their invention, nor are they intended to represent or imply that the experiments below are all of or the only experiments performed. Efforts have been made to ensure accuracy with respect to numbers used (e.g., amounts, temperatures, etc.), but some experimental error and deviation should be accounted for. Unless indicated otherwise, parts are parts by weight, molecular weight is weight average molecular weight, and temperature is in degrees Centigrade.

### EXAMPLE 1

Interaction of apoE with LRP and Effect on Neurite Outgrowth The following materials and methods were used to obtain the results discussed below.

Materials

Dimyristoylphosphatidylcholine (DMPC), DME/F12 (1:1 mixture of Dulbecco's nutrient modified Eagle's medium and Ham's mixture F12), media supplements (progesterone, putrescine, selenite, and transferrin), sodium chlorate, heparinase, lactoferrin, triolein, and egg yolk phosphatidylcholine (type XI-E) were purchased from Sigma Chemical Co. (St. Louis, Mo.), fetal bovine serum (FBS), and insulin from Gibco (Grand Island, N.Y.), suramin from Miles Inc. (FBA Pharmaceuticals, West Haven, Conn.), and DiI from Molecular Probes Inc. (Eugene, Oreg.). Neuro-2a was purchased from American Type Culture Collection (Rockville, Md.). Bovine CSF was obtained from Pel-Freez, Inc. (Fayetteville, Ark.).

Preparation of lipoproteins and Liposomes

Rabbit β-VLDL (d<1.006 g/ml) were isolated from the plasma of New Zealand white rabbits fed a high-fat, high-cholesterol diet for four days according to the method described by Kowal (1989) Proc. Natl. Acad. Sci. USA 86:5810-5814. Rabbit VLDL (d<1.006 g/ml) were isolated by ultracentrifugation from fasting plasma obtained from rabbits fed a normal rabbit chow. The VLDL were washed once by ultracentrifugation at d=1.006 g/ml. Bovine CSF lipoproteins (d<1.21 g/ml) were isolated by ultracentrifugation according to the method described by Pitas et al. (1987) J. Biol. Chem. 262:14352-14360. They were washed once by recentifugation through a solution of d=1.21 g/ml. Canine apoE HDL_{c} (d=1.006-1.02 g/ml) were isolated by ultracentrifugation and Pevikon electrophoresis from the plasma of foxhounds fed a semisynthetic diet containing hydrogenated coconut oil and cholesterol according to the method described by Mahley et al. (1977) Am. J. Pathol. 87:205-226. The β-VLDL were iodinated according to the method described by Bilheimer et al. (1972) Biochim. Biophys. Acta 260:212-221, and free iodine was removed by PD10 column chromatography.

The DMPC vesicles were prepared essentially according to the method described by Innerarity et al. (1979) J. Biol. Chem. 254:4186-4190. The DMPC alone (90 mg) or with the addition of cholesterol (10 mg) was dissolved in benzene and dried by lyophilization. The lyophilized material was then resuspended in 3 ml of 0.15 M NaCl, 10 mM Tris—Cl, and 1 mM EDTA (pH 7.6) and sonicated for 30 min at 37° C. using a sonifier cell disrupter (Branson 450, Danbury, Conn.) equipped with a microtip and full setting at 7 (50 watts). Innerarity (1979). The material was centrifuged for 10 min at 2,000 rpm (37° C.), and the supernatant was used for addition to cells. The lipid emulsion A was prepared according to the methods described Pittman et al. (1987) J. Biol. Chem. 262:2435-2442; and Spooner et al. (1988) J. Biol. Chem. 263:1444-1453. Briefly, the lipids were mixed together in the following ratio: 100 mg of triolein and 25 mg of egg yolk phosphatidylcholine and then dried under a stream of nitrogen. The pellet was then resuspended in 5 ml of 10 mM Tris—Cl, 0.1 M KCl, and 1 mM EDTA (pH 8.0) buffer and sonicated according to the method described by Spooner et al. (1988). The material was then centrifuged for 10 min at 2,000 rpm. The composition of the final emulsion was 2.7:1 for triolein:phosphatidylcholine (wt:wt). The size and morphology of the emulsion particles were determined by negative staining electron microscopy.

Preparation of Expression Vectors

The expression vectors were assembled in the pBSSK plasmid (Stratagene, La Jolla, Calif.). The constructs contained the rat neuron-specific enolase (NSE) promoter (kindly provided by Dr. J. G. Sutcliffe, Scripps Clinic and Research Foundation, La Jolla, Calif.), which has been previously used to direct neuron-specific expression of the human amyloid precursor protein and β-galactosidase in transgenic mice. Quon et al. (1991) Nature 352:239-241; and Forss-Petter (1990) Neuron 5:187-197. In addition, the construct contained the first exon (noncoding), the first intron, and the first six bases of the second exon (prior to the initiation methionine) of the human apoE gene, followed by the apoE cDNA.

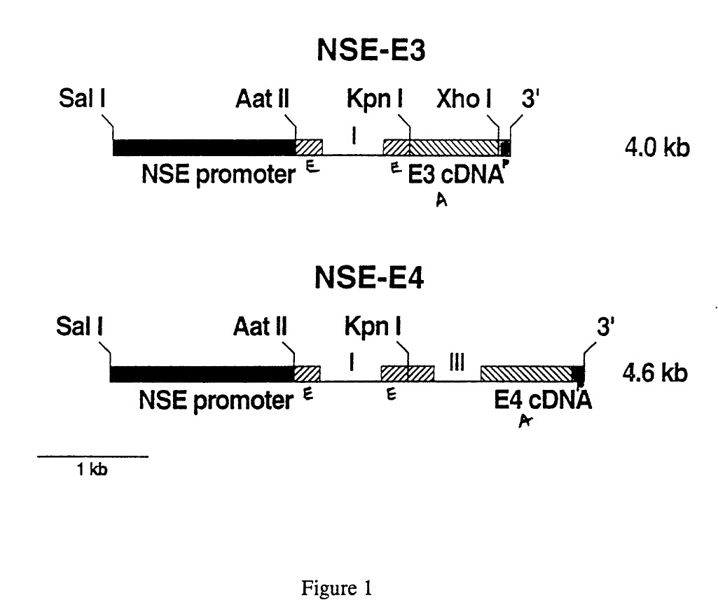

The apoE4 construct was identical except that it also contained the third intron (FIG. 1). The noncoding region of the fourth exon was downstream from the cDNA, followed by 112 bp of the 3′-flanking sequence of the human apoE gene that contains the polyadenylation signal. The apoE constructs for insertion in these expression vectors were kindly provided by Drs. S. Lauer and J. Taylor of the J. David Gladstone Institutes. The orientation of the cDNAs was confirmed by sequencing, using an Applied Biosystems automated sequencer. The final constructs were referred to as NSE-E3 (for apoE3 cDNA) and NSE-E4 (for apoE4 cDNA) (FIG. 1). Plasmid DNA was purified by two rounds of cesium chloride gradient ultracentrifugation according to the method described by Sambrook et al. (1989) Molecular Cloning: A Laboratory Manual, 2nd ed. Cold Spring Harbor Laboratory Press, Cold Spring Harbor, N.Y. To test the constructs, Chinese hamster ovary cells and human embryonic kidney 293 cells were transiently transfected (lipofectin-mediated), and the concentration of apoE in the medium was measured as described below. Similar levels of expression of apoE3 and apoE4 were achieved.

Production of Stably Transfected Neuro-2a Cell Lines

Cells at 20-30% confluence were cotransfected with pSV2neo and either NSE-E3 or NSE-E4 using a calcium phosphate precipitation protocol essentially as described by Chen et al. (1988) BioTechniques 6:632-638. Control cells were transfected with pSV2neo alone, following the same protocol. Stably transfected cells were selected by growth in DME/F 12 media containing 10% FBS and 400 Tg/ml of G418 (Geneticin, Gibco). Individual G418-resistant colonies were selected and expanded. Secretion of human apoE3 or apoE4 by the transfected cells was verified by Western blotting of the conditioned media.

ApoE Quantitation

Intracellular, cell-surface-bound, and secreted apoE were quantitated in cells maintained for 96 hr in N2 medium, a serum- and lipid-free medium (DME/F12 containing growth supplements as described in Bottenstein et al. (1980) Exp. Cell Res. 129:361-366), with or without added β-VLDL (40 Tg cholesterol per ml). The medium was changed once at 48 hr. The secreted apoE reported is that present in the medium following the second 48 hr incubation. The media were collected and, after the addition of protease inhibitors, centrifuged to eliminate suspended cells. The cell monolayers were washed with PBS and incubated for 1 hr at 4° C. with 2 ml of DMEM/F 12 containing 25 mM Hepes and 10 mM suramin, a polyanion that is able to release apoE bound to the cell surface. Ji et al. (1994). The apoE was precipitated from the medium and the suramin extract by addition of 50 Tg/ml of fumed silica (Sigma, St. Louis, Mo.) and centrifugation at 13,000 x g for 10 min.

Each pellet was washed three times with sterile water and dissolved in gel-loading buffer. Cellular apoE was extracted from the cells, following suramin removal of surface-bound apoE, using STEN buffer (50 mM Tris—Cl, pH 7.6, containing 150 mM NaCl, 2 mM EDTA, 1% NP-40, 20 mM PMSF, and 5 Tg/ml leupeptin). Samples were electrophoresed on 5-20% polyacrylamide gradient gels containing sodium dodecyl sulfate, according to the method described by Ji et al. (1994) J. Biol. Chem. 269:13429-13436. The proteins were transferred to nitrocellulose paper by blotting and treated with an anti-human apoE polyclonal antiserum (1:1,000 dilution) raised in rabbit (generously provided by Dr. K. H. Weisgraber, Gladstone Institutes). The nitrocellulose immunoblot was then incubated with donkey anti-rabbit secondary antibody conjugated to horseradish peroxidase (1:5,000 dilution) (Amersham, Arlington Heights, Ill.). After washing to remove unbound antibody, the immunocomplex was detected using an ECL kit (Amersham), according to the manufacturer's instructions. Quantitation of the level of apoE bound, internalized, and secreted by the cells was accomplished by densitometric scanning (Ambis Scanner, San Diego, Calif.) and based on a standard curve of purified human plasma apoE3 and apoE4.

Neurite Outgrowth

Cells were grown in DME/F12 containing 10% FBS and G418 (400 Tg/ml). On the day the experiment was initiated, the cells were subcultured into 35 mm plates in DME/F12 with 10% FBS. The cells were allowed to adhere to the plastic plates for 2 hr at 37° C., and then the culture medium was changed to N2 medium with or without increasing concentrations of lipoproteins. After 48 hr at 37° C., the media were replaced with the same medium (with or without lipoproteins), and the incubation was continued for an additional 48 hr. (The CSF lipoproteins were dialyzed against N2 medium prior to addition to the cells.) The cells then were washed with DME/F 12 containing 0.2% BSA, nonspecifically stained for 1 hr at 37° C. with DiI added in DMSO according to the method described by Nathan et al. (1994) Science 264:850-852, and fixed with 2.5% glutaraldehyde in PBS (v/v). Neurons were imaged in fluorescence mode with a confocal laser scanning system (MRC-600, BioRad, Hercules, Calif.), and the images were digitized with an Image-l/AT image analysis system (Universal Images, West Chester, Pa.). The neuronal images were coded before characterization, and the following variables were measured: 1) number of neurites (defined as cell surface projections at least one-half the cell diameter) on each neuron; 2) neurite branching (the number of branch points on each neurite); and 3) neurite extension (the length of the longest neurite, measured from the cell body). Typically, in each experiment the neurites of 20 to 40 neurons per plate were measured and the results preserved as the mean ±S.E.M.

In studies on the effect of the inhibitors of lipoprotein binding to the LRP, cells were incubated for 1 hr at 37° C. in N2 medium containing the indicated concentrations of either lactoferrin, chlorate, or heparinase or with the receptor-associated protein (RAP). Then the β-VLDL were added, and the incubation was continued for a total of 96 hr. The reagents, except for β-VLDL, were re-added every 24 hr. The media and β-VLDL were replaced after 48 hr.

Cell Association and Degradation of ¹²⁵I-β-VLDL

The cells were grown for 24 hr in 35 mm dishes in N2 medium alone. Then ¹²⁵I-β-VLDL (3 Tg of protein per ml of medium) were added, and the incubation was continued for 16 hr at 37° C. The medium was analyzed for TCA-soluble lipoprotein degradation products according to the method described by Goldstein et al. (1983) Met. Enzymol. 98:241-260. The cells were placed on ice, washed with PBS containing 0.2% BSA, and dissolved in 0.1 N NaOH. Lipoprotein cell association was determined by measuring cellular radioactivity using a gamma counter (Beckman Gamma 8000, Beckman Instruments, Fullerton, Calif.) and according to the method described by Goldstein et al. (1983).

Cell Association of DiI-labeled β-VLDL

The cells were grown for 24 hr in 35 mm dishes in N2 medium. Then DiI-labeled β-VLDL (4 Tg of protein per ml of medium), was prepared according to the methods described by Pitas et al. (1983) Arteriosclerosis 3:2-12; and Pitas et al. (1981) Arteriosclerosis 1:177-185, were added, and the incubation was continued for 5 hr at 37° C. The cells were then washed with PBS and fixed with 4% paraformaldehyde in PBS (v/v). Uptake of DiI-labeled β-VLDL was visualized by fluorescence microscopy. To quantitate the amount of DiI-labeled lipoprotein in the cells at the end of the incubation, the cells were scraped, using two 0.5 ml aliquots of PBS, and lyophilized. The DiI was extracted from the dried cell pellet with methanol and analyzed using a spectrofluorometer (excitation 520 nm, emission 570 nm). Pitas et al. (1983). Standards of DiI in methanol were used for quantitation.

Association of ApoE with Lipid Particles

ApoE3 and apoE4 were iodinated using Bolton-Hunter reagent (DuPont NEN, Boston, Mass.) according to the method described by Innerarity et al. (1983) J. Biol. Chem. 258:12341-12347, and then incubated with the lipid particles for 1 hr at 37° C. The samples were then fractionated by chromatography on a Superose 6 column (10/50 HR, Pharmacia Fine Chemicals, Uppsala, Sweden) and eluted with 1 mM EDTA in PBS at a constant flow rate of 0.5 ml/min. Fractions of 0.5 ml were collected and analyzed for cholesterol and triglyceride, and the ¹²⁵I-apoE content was measured in a Beckman 8000 counter (Beckman Instruments) and according to the method described by Dong et al. (1994) J. Biol. Chem. 269:22358-22365.

Statistical Analysis

Data were analyzed using a paired t-test.

Results

The levels of apoE secreted into the medium, bound to the cell surface, and accumulated intracellularly by the stably transfected Neuro-2a cells expressing human apoE3 or apoE4 were assessed by Western blot analysis and quantitated by densitometry. The results obtained are presented in Table. 1.

TABLE 1
ApoE3 or apoE4 secreted, releasable by suramin, or present inside
cells stably transfected with apoE3 or apoE4 cDNA

| | | Releasable | |
|---|---|---|---|
| | | ng of apoE/mg of | |
| Cells | Secreted | cell protein | Intracellular |
| ApoE3-expressing | | | |
| Clone #1 | 54 | 6.2 | 140 |
| +β-VLDL | 56 | 7.2 | 119 |
| Clone #3 | 44 | 4.9 | 259 |
| +β-VLDL | 45 | 4.3 | 251 |
| ApoE4-expressing | | | |
| Clone #4 | 60 | 6.7 | 215 |
| +β-VLDL | 63 | 5.3 | 231 |
| Clone #5 | 69 | 8.0 | 135 |
| +β-VLDL | 62 | 6.5 | 128 |
| Clone #6 | 89 | 5.2 | 111 |
| +β-VLDL | 87 | 5.6 | 105 |

To obtain the results depicted in Table 1, transfected cells were incubated for 96 hr in medium with or without β-VLDL (40 Tg cholesterol/ml). The medium was changed at 48 hr. ApoE secreted in the last 48 hr, intracellular, and suramin-releasable (surface-bound) apoE were quantitated at the end of the 96 hr of incubation as described in Nathan et al. (1995). The data are the mean of two separate determinations. The duplicates did not differ by more than 12%.

The results depicted in Table 1 indicate that the cells secreted 44-54 ng of apoE3 and 60-89 ng of apoE4 per mg of cell protein in 48 hr. The apoE3- and apoE4-secreting cells had similar amounts of apoE bound to the cell surface (releasable by suramin treatment), ranging from 4.9 to 8.0 ng of apoE per mg of cell protein. The intracellular content of apoE in the two apoE3-expressing cell lines was 140 and 259 ng of apoE per mg of cell protein. Similar amounts of intracellular apoE (111-215 ng/mg) were seen in the apoE4-expressing cell lines. The addition of β-VLDL to the cells did not have a significant effect on the amount of apoE secreted, surface-bound, or present within the apoE3- or apoE4-secreting cells (Table 1).

In initial experiments, two Neuro-2a cell lines that secreted similar amounts of apoE3 (clone 1, 54 ng/mg of cell protein) and apoE4 (clone 4, 60 ng/mg of cell protein) (Table 1) were used to examine neurite growth. When these cells were grown in N2 medium in the absence of β-VLDL, there were no apparent differences in neurite outgrowth between the apoE3- and apoE4-secreting cells. However, incubation of the cells in N2 medium containing β-VLDL resulted in a markedly different pattern in the neurite outgrowth from these cells. ApoE3-secreting cells incubated with β-VLDL developed long neurites, whereas in apoE4-secreting cells neurite outgrowth was suppressed.

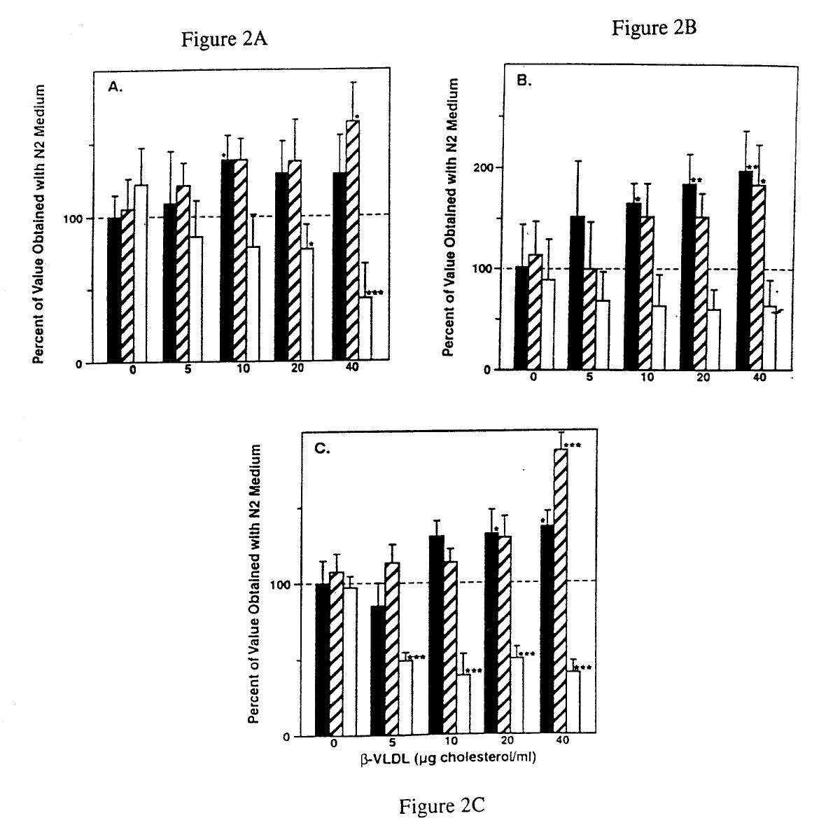

Differences in neurite outgrowth in the absence and presence of increasing concentrations of β-VLDL were quantitated by measuring the number of neurites per cell, neurite branching, and neurite extension (FIGS. 2A, B, and C, respectively). The values for the non-apoE transfected control cells incubated for 96 hr in N2 medium in the absence of β-VLDL are set at 100%. The expression of either apoE3 or apoE4 by the transfected Neuro-2a cells did not influence neurite number, branching, or extension when the cells were grown in N2 medium in the absence of added lipoprotein (FIGS. 2A, B, and C). To obtain the results depicted in FIG. 2, cells (clone #1 for apoE3-expressing and clone #4 for apoE4 expressing) were incubated for 96 hr in N2 medium alone or in medium containing increasing concentrations of β-VLDL. The media were changed at 48 hr. The cells were stained with DiI and fixed, and the indicated parameters were measured. Each data point was obtained by the measurement of 20-50 cells expressing neurites in four separate experiments. The data are presented as the percentage of the value obtained with control cells with N2 medium alone. The data are the mean ± the S.E.M. As depicted in FIG. 2, the average values obtained with control cells incubated with N2 medium alone were: A: neurites per cell=3; B: branch points per neurite=2; C: average neurite length=155 Tm.

For calculation of the level of significance for the effect of added β-VLDL, the results in the presence of β-VLDL are, compared to the data obtained with the same cells in the absence of β-VLDL (i.e., grown in N2 medium alone). *p<0.025; **p<0.010; ***p<0.005.

However, as shown in FIG. 2A, the addition of β-VLDL resulted in an increase in the number of neurons in the control cells and in the cells secreting apoE3 (significantly increased at 40 Tg of β-VLDL cholesterol/ml compared with apoE3-secreting cells in N2 medium). On the other hand, in the presence of high concentrations of β-VLDL, the Neuro-2a cells secreting apoE4 showed a significant reduction in the number of neurites per cell as compared with the apoE4-secreting cells in the N2 medium.

As previously described for DRG cells (Handelmann et al. (1992) J. Lipids Res. 33:1677-1688; and Nathan et al. (1994)), the addition of β-VLDL alone resulted in increased branching of neurites. As shown in FIG. 2B, addition of β-VLDL to the non-apoE-transfected cells resulted in a significant increase in neurite branching. In addition, at the highest concentration of β-VLDL cholesterol, the apoE3-secreting cells displayed enhanced branching by comparison with the apoE3-secreting cells grown in N2 medium alone. In contrast, the apoE4-secreting cells tended to show decreased branching when incubated with β-VLDL; however, this decrease did not reach statistical significance.

Neurite extension was increased in the Neuro-2a cells secreting apoE3 when they were incubated with the highest concentrations of β-VLDL. In contrast, in the apoE4-secreting cells neurite extension was very significantly suppressed even at the lowest concentration of β-VLDL used (FIG. 2C).

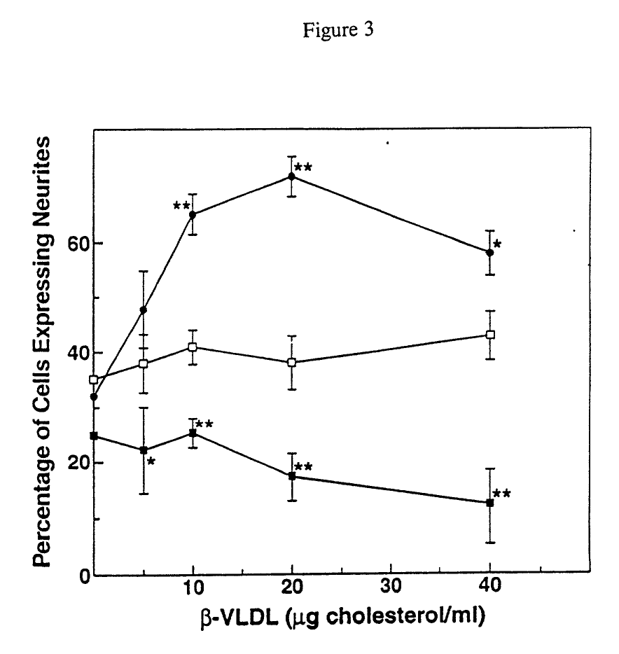

The results described in FIG. 2 were based on a comparison of cells having neuritic outgrowths and did not take into account those Neuro-2a cells without neuritic extensions. Approximately 25-30% of the Neuro-2a cells in N2 medium possessed neurite extensions (defined as a cell-surface projection of at least one-half the cell diameter). However, as shown in FIG. 3, it was apparent that in the presence of β-VLDL, the number of apoE3 -secreting cells developing neurites increased markedly to 60-70% of the total. On the other hand, the number of apoE4-secreting cells developing neuritic extensions was significantly reduced, compared with the control or apoE3-secreting cells. Thus, the apoE3-secreting cells incubated with β-VLDL not only had longer neuritic extensions but also showed an increase in the number of cells with neurites. The apoE4-secreting cells grown in the presence of β-VLDL showed fewer neurites, and those that were produced were much shorter.

To ensure that the differential effect of β-VLDL on neurite outgrowth in the apoE3- and apoE4-secreting cells was not due to clonal variation or to differences in the secretion or intracellular content of apoE in the various cell lines, additional experiments were performed with the other stably transfected cell lines secreting apoE3 or apoE4. Incubation of these cells with β-VLDL also resulted in differential effects of apoE3 and apoE4 on neurite outgrowth. The results obtained are presented in Table 2.

TABLE 2
Effect of β-VLDL (40 Tg cholesterol/ml medium) on the number of
neurites per cell, neurite branching, and neurite extension from cells
stably transfected with apoE3 or apoE4

| | | |
|---|---|---|
| Number of Neurites | Branching | Extension |

| | |
|---|---|
| Cell type | (% of values obtained with control cells in N2 medium alone) |

ApoE3-expressing

| | | | |
|---|---|---|---|
| Clone #1 | 165 ± 30 | 186 ± 39 | 186 ± 13 |
| Clone #2 | 150 ± 25 | 180 ± 15 | 190 ± 23 |
| Clone #3 | 170 ± 39 | 175 ± 20 | 180 ± 25 |

ApoE4-expressing

| | | | |
|---|---|---|---|
| Clone #4 | 43 ± 25 | 65 ± 26 | 41 ± 9 |
| Clone #5 | 49 ± 15 | 70 ± 31 | 50 ± 15 |
| Clone #6 | 53 ± 19 | 60 ± 25 | 45 ± 19 |

In Table 2, the level of secretion of apoE by clones #1, #3, #4, #5, and #6 is as described for Table 1. Clone #2 secreted 36 ng of apoE3/mg of cell protein/48 hr. Surface-bound and internalized apoE was not quantitated for clone #2. The conditions for incubation with β-VLDL are as described for FIG. 2. Each data point was obtained by the measurement of 25-40 cells. The data are the mean ± S.E.M.

As summarized in Table 2, in the presence of β-VLDL, all of the apoE4-secreting cells showed a significant reduction in the number of neurites expressed, branching, and neurite extension, whereas the apoE3-secreting cells displayed an increased number of neurites, increased branching, and increased extension as compared to cells grown in N2 medium lacking a source of lipoprotein.

To determine whether apoE4 blocks neurite extension in the presence of β-VLDL or whether it induces neurite retraction, the cells were incubated for 48 hr in N2 medium alone to stimulate neurite outgrowth. The medium was changed, and the cells incubated for an additional 48 or 96 hr in media with β-VLDL (40 Tg of cholesterol per ml). The addition of β-VLDL did not decrease the extension of neurites of apoE4-expressing cells compared with cells incubated in N2 medium alone. Therefore, apoE4 in the presence of β-VLDL, inhibits neurite extension directly and does not cause a retraction of neurites that have already extended.

Other lipoproteins were used to determine if any lipid vehicle carrying apoE would substitute for β-VLDL. Incubation of the apoE3- or apoE4-expressing cells with rabbit VLDL, a lipoprotein rich in triglyceride, resulted in similar effects on neurite extension as obtained with β-VLDL. The results are presented in Table 3.

TABLE 3
Effect of β-VLDL, VLDL or lipid emulsions on neurite extension
from cells stably transfected with apoE3 or apoE4 cDNA

| | Lipid | | | ApoE3- | apoE4- |
|---|---|---|---|---|---|
| | comp- | Mean | Control | expressing | expressing |
| | osition | Size | % of value obtained with control | | |
| Treatment | (wt/wt/wt) | (nm ± S.D.) | cells in N2 medium alone | | |
| N2 alone | , | , | 100 ± 10 | 110 ± 15 | 115 ± 11 |
| β-VLDL | CHOL: | 43.7 ± 25.6 | 120 ± 15 | 160 ± 18^{a} | 60 ± 13^{a} |
| | Tg:PL | | | | |
| | (5.6:0.4:1) | | | | |
| VLDL | CHOL: | 39.5 ± 18.7 | 110 ± 11 | 155 ± 21^{a} | 61 ± 19^{a} |
| | Tg:PL | | | | |
| | (1:7.4:1) | | | | |
| Emul A | Tg:PL | 35.8 ± 14.9 | 95 ± 14 | 150 ± 12^{a} | 75 ± 12^{a} |
| | (2.7:1) | | | | |

To obtain the results depicted in Table 3, cells (clone #1 for apoE3 -expressing and clone #4 for apoE4-expressing) were incubated for 96 hr in N2 medium alone or containing the indicated concentrations of particles: β-VLDL, 40 Tg cholesterol/ml medium (this corresponds to 5 Tg triglyceride/ml medium); VLDL, 5 Tg triglyceride/ml medium; emulsion A, 5 Tg triglyceride/ml medium. CHOL=cholesterol; Tg=triglyceride; PL=phospholipid. Each data point was obtained by the measurement of 30, 40 cells expressing neurites in three separate experiments. The data are the mean ± S.E.M. ^{a}p<0.010 versus control***.

As shown in Table 3, when the Neuro-2a cells secreting apoE3 were incubated with VLDL, they showed an increase in neurite extension, whereas the apoE4-secreting cells in the presence of VLDL showed an inhibition of neurite extension. In other experiments, human LDL and canine apoE HDL_{C}, an apoE-enriched plasma high density lipoprotein (HDL) induced by cholesterol feeding and resembling apoE-containing lipoproteins in the CSF (Pitas et al. (1987)), also were used. The apoE3- and apoE4-secreting Neuro-2a cells did not respond to LDL (40 Tg cholesterol/ml) (i.e., there was no difference in neurite extension as compared with control cells grown in N2 medium alone). On the other hand, incubation of apoE HDL_{C} (40 Tg cholesterol/ml) with the apoE4-secreting or apoE3-secreting cells resulted in only a small reduction or increase in neurite extension, respectively (control cells in N2 medium, 100%; apoE4-secreting cells plus HDL_{C}, 85, 90% of the value obtained with N2 medium; apoE3-secreting cells plus HDL_{c,} 110% of the value obtained with N2 medium).

Liposomes and lipid emulsions also were used in an attempt to define the type of lipid vehicle required for the delivery of the apoE. The DMPC emulsion alone or DMPC complexed with cholesterol were incubated with the apoE3- and apoE4-secreting cells for 96 hr at increasing phospholipid concentrations of up to 45 Tg phospholipid and 5 Tg cholesterol/ml medium (higher concentrations were toxic to the cells).

In these studies, there was no effect on neurite outgrowth with either of the apoE-transfected Neuro-2a cells. Previously, it was shown that apoE complexes with DMPC and mediates high-affinity binding to the LDL receptor. Pitas et al. (1980) J. Biol. Chem. 255:5454-5460. On the other hand, a lipid emulsion particle (emulsion A in Table 3), which was a triglyceride- and phospholipid-containing spherical particle (approximately 35.8 nm), caused a significant enhancement of neurite extension in the apoE3-secreting cells and was associated with an inhibition of outgrowth in the apoE4-secreting cells. Thus, specific combinations of lipids and/or a unique particle size may be required to elicit the apoE isoform, specific effects on neurite outgrowth. It is interesting to note that the delivery of cholesterol to the cells does not appear to be required for the differential effect.

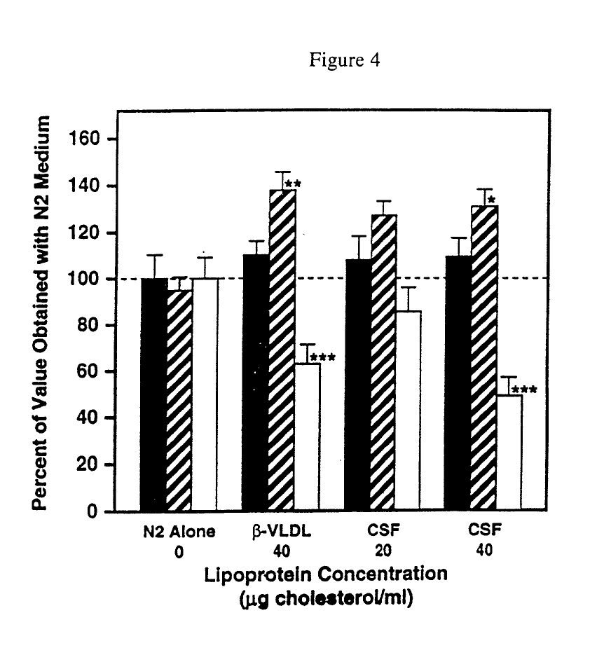

Additional studies using the lipoproteins from bovine CSF suggest that natural lipoproteins in the CNS may mediate the isoform-specific effects of apoE3 and apoE4. As shown in FIG. 4, addition of lipoproteins isolated from CSF (d<1.21 g/ml) to the cells caused an inhibition of neurite outgrowth from the apoE4-expressing cells and an increase in outgrowth from the apoE3-expressing cells. When CSF lipoproteins were used at a concentration of 40 Tg lipoprotein cholesterol/ml, the effect was similar to that obtained using β-VLDL at the same concentration.

CSF lipoproteins (d<1.21 g/ml) were analyzed for protein and cholesterol content and apolipoprotein composition. The ratio of cholesterol to protein was approximately 1:1, similar to data reported for canine CSF. Pitas et al. (1987). The bovine CSF lipoproteins (d<1.21 g/ml) contained only apoE and apoA-I when separated by sodium dodecyl sulfate polyacrylamide gel electrophoresis and visualized by Coomassie Brilliant Blue staining. These results are similar to those reported previously for human and canine CSF lipoproteins. Pitas et al. (1987); and Roheim et al. (1979) Proc. Natl. Acad. Sci. USA 76:4646-4649.

The ability of the neuroblastoma cells to bind, internalize, and degrade β-VLDL was examined to determine whether the differences in neurite outgrowth in the apoE3- and apoE4-expressing cells was due to a different ability of the secreted apoE3 and apoE4 to stimulate the delivery of apoE and/or lipoprotein lipids to the cells. In these studies, ¹²⁵I-β-VLDL were used to quantitate the binding, uptake, and degradation of the lipoproteins in the Neuro-2a cells. The results are presented in Table 4.

TABLE 4
Cell association and degradation of ¹²⁵I-β-VLDL by
stably transfected and control cells

¹²⁵I-β-VLDL

| | Cell association | Degradation |
|---|---|---|
| Cell type | (ng of lipoprotein protein/mg of cell protein) | |
| Control cells | 750 ± 16 | 2,467 ± 331 |
| ApoE3-expressing cells | 671 ± 40^{a} | 1,945 ± 219 |
| ApoE4-expressing cells | 662 ± 50^{a} | 1,788 ± 188^{b} |

To obtain the results depicted in Table 4, cells were incubated for 24 hr in N2 medium alone. The ¹²⁵I-β-VLDL (3 Tg protein/ml medium) were then added, and after 16 hr at 37° C. the lipoprotein cell association (bound and internalized) and degradation by Neuro-2a cells were measured. The data reported are the mean of two separate experiments performed in duplicate (±S.D.). Control=cells transfected with pSV2neo alone. In Table 4, a represents <0.05 versus control and b represents <0.01 versus control.

The results presented in Table 4 indicate that the total amount of cell-associated (bound and internalized) ¹²⁵I-β-VLDL was very similar in the apoE3- and apoE4-secreting cells (both were slightly lower than that seen in the non-apoE-transfected control cells). The degradation of ¹²⁵I-β-VLDL by the apoE3- and apoE4-secreting cells was similar. There was a small (but statistically significant) decrease in the degradation of ¹²⁵I-β-VLDL by the apoE4-secreting cells when compared with the non-apoE-transfected control Neuro-2a cells.

In a parallel experiment, the cells were incubated with DiI-labeled β-VLDL to visualize the internalization of the lipoproteins in the apoE3- and apoE4-secreting cells by fluorescence microscopy. Following internalization, DiI is trapped in the lysosomes, and the fluorescent intensity of the cells, therefore, is proportional to the total amount of lipoprotein internalized and degraded. Pitas et al. (1983). In these studies, no difference in the uptake of DiI-labeled β-VLDL was observed in the apoE3- and apoE4-secreting cells. Extraction and quantitation of the DiI from cells incubated with DiI-labeled β-VLDL (40 Tg of cholesterol per ml) for 16 hr at 37° C. confirmed the visual impression that the uptake of DiI-labeled β-VLDL was similar in the apoE3- and apoE4-secreting cells. The control cells incorporated 8.9±0.4 ng of DiI per mg of cell protein, while the apoE3- and apoE4-expressing cells incorporated 10.2±1.0 and 10.8±0.3 ng of DiI per mg of cell protein, respectively.

To demonstrate that apoE binds to the lipid particles when it is present at the concentrations secreted by the cells, radiolabeled apoE3 or apoE4 was incubated with the β-VLDL, VLDL, or emulsion A for 1 hr at 37° C. (100 ng of apoE with 40 Tg of β-VLDL cholesterol or 100 ng of apoE with either 5 Tg of VLDL or emulsion A triglyceride) and fractionated by FPLC. Approximately 70% of the apoE was associated with the β-VLDL and 50% with the VLDL and emulsion A. There was no difference in the amount of apoE3 or apoE4 associated with the lipid particles.

### EXAMPLE 2

### Specific Inhibition of apoE Binding to apoE Binding Receptor

To determine which receptor was involved in mediating the differential effects of apoE3 and apoE4 on neurite outgrowth, inhibitors that block the binding and internalization of apoE-enriched lipoproteins by the HSPG-LRP pathway, but not by the LDL receptor pathway, were used. The effect on neurite outgrowth was then determined. Prior to the addition of β-VLDL, the cells were preincubated for 1 hr with either heparinase (20 units/ml) and chlorate (20 mM), with the RAP (5 Tg/ml), or with lactoferrin (10 Tg/ml). The binding of apoE-enriched lipoproteins to the LRP requires their initial binding to cell-surface HSPG. Heparinase and chlorate cleave and reduce the sulfation of cell-surface HSPG, respectively. Ji et al. (1993) J. Biol. Chem. 268:10160-10167; and Humphries et al. (1989) Met. Enzymol. 179:428-434. Lactoferrin blocks binding of lipoproteins to both HSPG and LRP, whereas the RAP primarily blocks the binding of apoE-enriched lipoproteins to the LRP. All of these reagents previously have been shown to inhibit the uptake of apoE-enriched β-VLDL by the LRP. Mahley et al. (1994) Ann. N.Y. Acad. Sci. 737:39-52; Ji et al. (1993); Ji et al. (1994a); and Willnow et al. (1992) J. Biol. Chem. 267:26172-26180. As previously shown in FIG. 2, β-VLDL alone stimulated the outgrowth of neurites. The stimulation of neurite outgrowth by β-VLDL was further enhanced in the apoE3 -expressing cells and markedly inhibited in the apoE4-secreting cells (Table 5).

TABLE 5
Effect of chlorate, heparinase, the RAP, and lactoferrin in the presence of
β-VLDL on neurite extension from cells stably transfected with
apoE3 or apoE4 cDNA

| | Control | ApoE3-expressing | ApoE4-expressing |
|---|---|---|---|
| | % of value obtained with control | | |
| Treatment | cells in N2 medium alone | | |
| N2 alone | 100 ± 8 | 105 ± 10 | 103 ± 9 |
| β-VLDL (40 Tg | 160 ± 13 | 209 ± 13^{a} | 70 ± 4^{b} |
| cholesterol/ml) | | | |
| β-VLDL + | 159 ± 14 | 163 ± 20^{c} | 138 ± 12 |
| chlorate (20 mM) | | | |
| and heparinase | | | |
| (20 units/ml) | | | |
| β-VLDL + RAP | 176 ± 11 | 179 ± 15 | 160 ± 16 |
| (5 Tg/ml)^{d} | | | |
| β-VLDL + | 128 ± 16 | 154 ± 19^{c} | 130 ± 12 |
| lactoferrin | | | |
| (10 Tg/ml) | | | |

To obtain the results depicted in Table 5, cells were incubated for 1 hr in N2 medium alone or containing the indicated concentrations of chlorate, heparinase, RAP, or lactoferrin. Then the β-VLDL were added, and the incubation was continued for a total of 96 hr. The reagents, except for β-VLDL, were re-added every 24 hr. The media and β-VLDL were changed at 48 hr. Each data point was obtained by measuring 30, 40 neurons expressing neurites in two separate experiments. Data are the mean ± S.E.M. ^{a}p<0.05, ^{b}p<0.01 versus value obtained with control cells (non-apoE-expressing cells incubated with β-VLDL). ^{c}p<0.05 versus apoE3-expressing cells with β-VLDL alone. ^{d}In a parallel set of experiments, 5 Tg/ml of RAP did not block the binding of DiI-labeled LDL to the Neuro-2a cells.

The results depicted in Table 5 indicate that the addition of chlorate and heparinase or the RAP did not block the stimulatory effect of β-VLDL on neurite outgrowth in the control cells (Neuro-2a cells not expressing apoE), suggesting that the effect of β-VLDL alone is mediated by the LDL receptor; however, these reagents blocked the isoform-specific effects in the cells secreting apoE (Table 5). Chlorate and heparinase treatment of the cells or the addition of the RAP prevented the stimulation of neurite extension in the apoE3-expressing cells incubated with β-VLDL (that is, significantly decreased the β-VLDL, induced neurite extension in the Neuro-2a cells secreting apoE3). Moreover, chlorate and heparinase or the RAP blocked the inhibition of neurite extension seen in the apoE4-expressing cells (that is, the apoE4-expressing cells in the presence of β-VLDL did not demonstrate inhibition of neurite extension but, in fact, showed increased extension) (Table 5). In the presence of heparinase and chlorate or the RAP, in the apoE-secreting cells, neurite outgrowth was similar to that observed when β-VLDL were added to the control cells in the absence of apoE (Table 5). Therefore, in the presence of these reagents, the LDL receptor, mediated effect of β-VLDL was not blocked. Lactoferrin also blocked the effects of apoE3 and apoE4 on neurite outgrowth; however, it also slightly suppressed the effect of β-VLDL on neurite extension in the control cells. These data show that inhibition of the interaction between β-VLDL and the HSPG-LRP pathway prevents the differential effects of apoE3 and apoE4 on neurite outgrowth (Table 5).

In dorsal root ganglion or neuroblastoma cells, apoE3 plus a source of lipid supports and facilitates neurite extension. ApoE3 appears to accumulate widely in cell bodies and neurites, stabilize the cytoskeleton and support neurite elongation, and directly or indirectly modulate microtubule assembly. ApoE4, on the other hand, does not appear to accumulate within neurons or support neurite extension, and may even destabilize the microtubule apparatus. The apoE4 effect appears to be mediated via the LRP pathway. Individuals with apoE4 clearly have normal neuronal development early in life. However, apoE4 may exert its detrimental effects later in life, by not allowing or supporting remodeling of synaptic connections. This affect is believed to be important in the pathogenesis of Alzheimer's disease because apoE4 is believed to contribute to Alzheimer's disease by aiding the formation of dense, complicated, possibly toxic plaques of Aβ peptide.

### EXAMPLE 3

### Methods of Detection of Agents That Interfere with the ApoE4 Domain Interaction

ApoE4 is iodinated using the Bolton-Hunter reagent (New England Nuclear Corp., Boston, Mass.) as previously described by Innerarity et al. (1979) J. Biol. Chem. 254:4186-4190, with specific activities ranging from 200 to 1100 dpm/ng. The iodinated apoE4 (0.5-2 mg in 50-10 ml 0.1 M NH₄HCO₃) is incubated with the test reagent or compound and the mixture is added to 250 ml of plasma from normal subjects at 37° C. for 2 h. Plasma is then fractionated into the various lipoprotein classes by chromatography on a Superose 6 column (10/50 HR, Pharmacia Fine Chemicals, Uppsala, Sweden) eluted with 20 mM sodium phosphate (pH 7.4), containing 0.15 M NaCl. The column flow rate is 0.5 ml/min, 0.5 ml fractions are collected, and the ¹²⁵I content is determined in a Beckman 8000 gamma counter (Beckman Instruments, Fullerton, Calif.). Reagents that interfere with apoE4 domain interaction will shift the preference of the “modified” apoE4 from VLDL to HDLs, resulting in a distribution that resembles that of apoE3 (run in parallel as a control).

### ApoE Metabolism

The metabolism of apoE-enriched β-VLDL by cultured neurons (Neuro-2a cells) was examined in three ways:

(1) by measuring the cell association (binding and internalization) of apoE-enriched ¹²⁵I-β-VLDL;

(2) by examining the metabolism of apoE-enriched DiI-labeled β-VLDL (DiI serving as a fluorescent marker for the lipid moieties of the lipoprotein particle); and

(3) by quantitating the ability of the apoE-enriched β-VLDL to increase the content of cellular cholesterol.

### Materials and Methods for Examples 4-6

Heparinase I and specific phospholipase C were purchased from Sigma Chemical Company (St. Louis, Mo.). Suramin was obtained from Research Biochemicals International (Natick, Mass.). Purified human plasma apoE and sheep anti-human apoE antibody were provided by Dr. Karl Weisgraber (Gladstone Institute of Cardiovascular Disease, San Francisco, Calif.). Donkey anti-sheep IgG was purchased from Jackson ImmunoResearch Laboratories, Inc. (West Grove, Pa.).

Preparation of Lipoproteins

Rabbit β-VLDL (d<1.006 g/ml) were isolated from the plasma of New Zealand White rabbits fed a high-fat, high-cholesterol diet for 4 days. The ratio of cholesterol to protein in this β-VLDL ranged from ˜15 to 20:1. Human apoE-enriched β-VLDL were prepared by incubating apoE with β-VLDL at 37° C. for 1 h. For some experiments, the apoE-enriched β-VLDL were reisolated by fast-performance liquid chromatography as follows. Either ¹²⁵I-β-VLDL and unlabeled apoE or ¹²⁵I-apoE and unlabeled β-VLDL were mixed in a 1:1.5 ratio of β-VLDL protein to apoE and incubated at 37° C. for 1 h. The mixture (250 μl) was then fractionated by chromatography on a Superose 6 column (Pharmacia Fine Chemicals, Uppsala, Sweden, 10/50 HR). The flow rate was 0.5 ml/min, and 0.5 ml fractions were collected. The elution profile was monitored by quantitation of ¹²⁵I and cholesterol.

Labeling of Lipoproteins and ApoE

The β-VLDL were iodinated by the method of Bilheimer et al. (1972) Biochim. Biophys. Acta. 260:212-221. Apolipoproteins E3 and E4 were iodinated by the Bolton-Hunter procedure (Bolton et al. (1973) Biochem. J. 133:529-539). Free iodine was removed by P10 column chromatography. The β-VLDL were labeled with 1,1′-dioctadecyl-3,3,3′,3′-tetramethylindocarbocyanine (DiI), as previously described (Pitas et al. (1981) Arteriosclerosis 1:177-185).

Detection of Intact ApoE in Cell Extracts

Murine neuroblastoma (Neuro-2a) cells were grown to ˜100% confluence in Dulbecco's modified Eagle's medium (DMEM)/F12 (1:1) containing 10% fetal bovine serum (FBS), washed with N2 medium, and incubated in N2 medium with β-VLDL (40 μg cholesterol/ml) alone or together with 30 μg/ml of iodinated apoE3 or iodinated apoE4. At the times indicated, the surface-bound apoE was removed by incubation with 10 mM suramin for 30 min at 4° C. The cells were then washed three times with phosphate-buffered saline (PBS) at 4° C. and gently scraped with a rubber policeman. The cells were dissolved in sodium dodecyl sulfate (SDS)-sample buffer, and the cell proteins were separated by 3-20% SDS-polyacrylamide gel electrophoresis (PAGE) and transferred to nitrocellulose membranes; apoE was detected by autoradiography.

Cell Culture

Neuro-2a cells were maintained in DMEM/F12 (1:1) containing 10% FBS; this medium was replaced with serum-free medium ˜16 h before use. Human skin fibroblasts were grown in DMEM containing 10% FBS. The LDL receptor-negative fibroblasts were grown in minimal essential medium supplemented with 10% FBS. Human hepatoma (HepG2) cells were maintained in minimal essential medium containing 10% FBS, 1% human nonessential amino acids, and 1% sodium pyruvate as described (Ji et al. (1994) J. Biol. Chem. 269:2764-2772). Mutant Chinese hamster ovary (CHO) cells pgsA-745 (xylose transferase-deficient), which do not produce any glycosaminoglycans, and pgsD-677 (N-acetylglucosamine transferase-deficient and glucuronic acid transferase-deficient), which do not produce heparin sulfate (Esko (1991) Curr. Opin. Cell Biol. 3:805-816) were kindly provided by Dr. J.D. Esko (University of Alabama, Birmingham). The CHO cells were maintained in F12 medium containing 7.5% FBS. Mouse LRP-negative (LRP^{-/-}) and LRP heterozygous fibroblasts (LRP^{+/-}), provided by Dr. J. Herz (University of Texas Southwestern Medical School, Dallas, Tex.), were maintained in DMEM containing 10% FBS. The cholesterol content of the β-VLDL or cultured cells was assayed.

Immunocytochemistry

Neuro-2a cells or fibroblasts grown in tissue culture dishes were washed with serum-free medium and incubated at 37° C. with apoE3 (30 μg/ml) or apoE4 (30 μg/ml) plus β-VLDL (40 μg of cholesterol/ml for the time indicated. After incubation, the cells were placed immediately on ice and washed with phosphate buffer. Cells were then fixed with 3% paraformaldehyde in 0.1 M phosphate buffer (pH 7.4) for immunofluorescence cytochemistry. Immunofluorescence from apoE was detected. The intensity of apoE immunofluorescence was quantitated by confocal microscopy.

Cell Association, Internalization, and Degradation of ApoE plus β-VLDL

Cultured cells were grown to ˜100% confluence, washed twice with fresh serum-free medium, and incubated at 37° C. with apoE-enriched β-VLDL. Before addition to the cells, the β-VLDL and apoE were incubated together (5 and 7.5 μg of protein, respectively, unless otherwise indicated) for 1 h at 37° C. Some cells were incubated with 50 μM chloroquine, and inhibitor of lysosomal protease, at 37° C. for 2 h before addition of the apoE-enriched β-VLDL. At the times indicated, the cells were placed on ice, and the medium was assayed for protein degradation products. For the cell association studies, Neuro-2a cells were washed five times on ice with 0.1 M PBS containing 0.2% bovine serum albumin and once with 0.1 M PBS. Cell-associated ligand represents both bound and internalized material. The fibroblasts were washed three times with DMEM-Hepes on ice and incubated with 10 mM suramin at 4° C. for 30 min to remove surface-bound ligand. The radioactivity remaining within the cells represents that which was “internalized.” After washing, the cells were dissolved in 0.1 N NaOH for measurement of radioactivity and protein concentration.

Internalization of ¹²⁵I-apoE-enriched β-VLDL by fibroblasts and by Neuro-2a cells was also studied at 18° C. The cells were placed in an 18° C. incubator for 20 min before the addition of the lipoproteins and then incubated for an additional 3 h at 18° C. After incubation, the cells were placed on ice, washed three times with DMEM-Hepes, and incubated with 10 mM suramin at 4° C. for 30 min to remove cell surface-bound ¹²⁵I-apoE. Degradation products of ¹²⁵I-β-VLDL or ¹²⁵I-apoE in the medium were assayed.

Uptake of DiI-labeled β-VLDL by Cultured Cells

Neuro-2a cells were incubated for 2 h at 37° C. with DiI-labeled β-VLDL alone or together with either apoE3 or apoE4. The cells were then washed and solubilized with 0.1 N NaOH, and the cell-associated DiI, which is proportional to the total amount of lipoprotein metabolized (bound, internalized, and degraded), was assayed.

Heparinase and Specific Phospholipase C Treatment of Cells

The cells were pretreated at 37° C. with heparinase I (10 units/ml) for 1 h or with specific phospholipase C (5 units/ml) for 30 min. The cells were then incubated in the presence of the enzymes with β-VLDL together with either apoE3 or apoE4. The β-VLDL (5 μg protein/ml) and apoE (7.5 μg/ml) were mixed and incubated together for 1 h at 37° C. before addition to the cells.

Pulse Chase of ¹²⁵I-apoE+β-VLDL by Wild-type and HSPG-deficient CHO Cells

Cultured cells were grown to ˜100% confluence, placed on ice, and washed twice with cold DMEM-Hepes. The cells were then incubated with ¹²⁵I-apoE+β-VLDL at 4° C. for 1 h to allow for cell-surface binding (zero time bound ligand). Cells were rinsed three times with cold F12 medium to remove unbound ligands. Prewarmed F12 medium was added, and the cells were incubated at 37° C. for the times indicated. At each point, the cells were again placed on ice, and the culture medium was collected. To 0.5 ml of medium was added 0.4 ml of 0.2% bovine serum albumin (Sigma) and 0.4 ml of 50% trichloroacetic acid (TCA). The medium was then incubated at 4° C. for 30 min and centrifuged at 3,000 rpm for 10 min. The supernatant was collected for ¹²⁵I-apoE degradation assay, and the pellet was counted as TCA-precipitable intact ¹²⁵I-apoE. the cells were washed once with cold DMEM-Hepes, incubated with 10 mM suramin on ice in a cold room for 30 min, and then dissolved in 0.1 N NaOH. Cellular radioactivity (internalized apoE) was measured with a gamma counter, and protein concentration was determined by Lowry's method.

### EXAMPLE 4

### Binding and Internalization of ApoE-enriched β-VLDL Particles

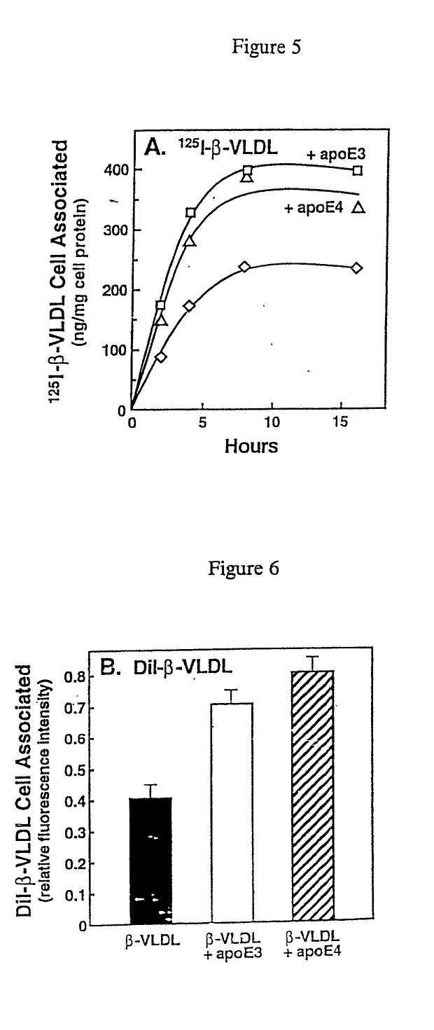

The cell association of ¹²⁵I-β-VLDL or ¹²⁵I-β-VLDL enriched with either human apoE3 or apoE4 by Neuro-2a cells was examined at 37° C. (FIG. 5). In these studies, the maximal cell association of β-VLDL alone was ˜225 ng/mg cell protein. The cell association of β-VLDL was enhanced ˜1.7-fold by apoE3 or apoE4. There was therefore no major isoform-specific difference in the ability of apoE3 or apoE4 to promote the binding and internalization of ¹²⁵I-β-VLDL, suggesting that similar amount of β-VLDL was internalized. In addition, DiI-labeled β-VLDL were used to examine the uptake of the β-VLDL particles by Neuro-2a cells (FIG. 6). DiI internalized with lipoproteins is retained by cells and can be used to quantitate the total amount of lipoprotein metabolized (bound, internalized, and degraded). In these studies, at 2 h both apoE3 and apoE4 stimulated the uptake of DiI-labeled β-VLDL (˜1.8-2-fold) compared with the amount of DiI-labeled β-VLDL internalized in the absence of apoE [apoE4 stimulated β-VLDL uptake to a slightly greater extent than apoE3 (p<0.002)].

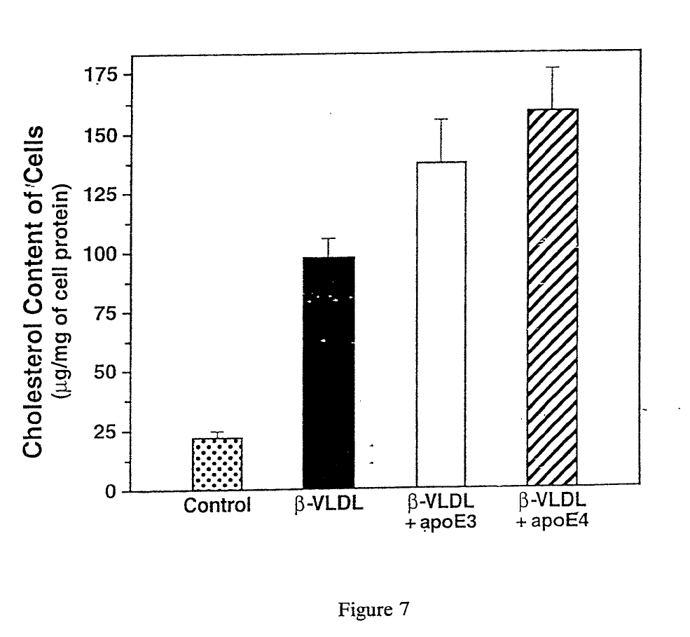

To establish further that apoE3 and apoE4 stimulated similar β-VLDL particle uptake, the cells were incubated in medium alone, medium containing β-VLDL, or medium containing β-VLDL and either apoE3 or apoE4, and the cholesterol content of the cells was determined (FIG. 7). The β-VLDL alone increased the cellular cholesterol content ˜4.7-fold, compared with the control cells maintained in the absence of lipoprotein. The β-VLDL enriched with either apoE3 or apoE4 increased the cellular cholesterol content [˜1.5-fold and ˜1.7-fold, respectively; the cholesterol content with apoE4 was significantly greater (p<0.005)] compared with the cells incubated with β-VLDL alone. Free apoE3 or apoE4 added without lipid had essentially no effect on the cellular cholesterol level. Taken together, the results examining the effect of apoE3 and apoE4 on the uptake of ¹²⁵I-β-VLDL or DiI-labeled β-VLDL and the ability of the cells to accumulate β-VLDL-derived cholesterol demonstrate that apoE3 and apoE4 stimulate β-VLDL internalization to a similar extent in Neuro-2a cells, with apoE4 being somewhat more active. Differences in lipoprotein particle uptake could not therefore account for the difference in the accumulation of apoE3 versus apoE4 (apoE3 greater than apoE4) in Neuro-2a cells incubated with apoE-enriched β-VLDL.

### EXAMPLE 5

### Intracellular Accumulation of ApoE Isoforms

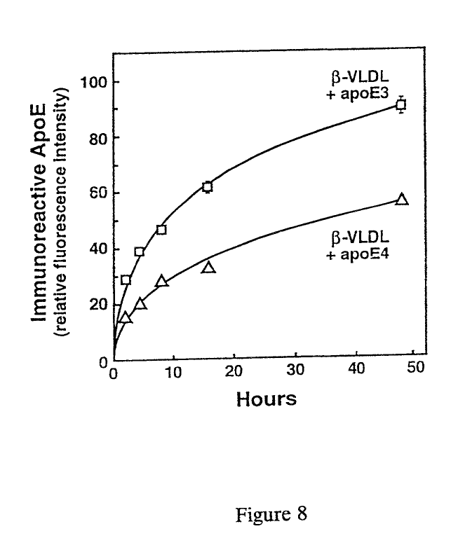

The time course for differential accumulation of apoE3 and apoE4 was analyzed in the Neuro-2a cells (FIG. 8). The cells were incubated with apoE-enriched β-VLDL for 2 to 48 h, permeabilized, and processed for immunocytochemistry with a polyclonal antibody that detects purified human apoE3 and E4 equally well on western blots. Immunoreactive apoE was detected and quantitated by confocal microscopy to measure the relative fluorescence intensity. At the earliest time point (2 h), the cells contained approximately 1.8-fold more apoE3 than apoE4. This difference in the level of immunoreactive apoE was maintained for up to 48 h (˜1.6-fold more apoE3 than apoE4) (FIG. 8).

The accumulated intracellular apoE was primarily intact protein. Cells were incubated with apoE-enriched β-VLDL for the times indicated; the cellular proteins were extracted, resolved by SDS-PAGE, and transferred to nitrocellulose, and apoE was detected by autoradiography. Autoradiography demonstrated a greater cellular accumulation of apoE3 than apoE4 and no obvious accumulation of degradation products. Western blot analysis yielded similar results, revealing the differential intracellular accumulation of intact apoE.

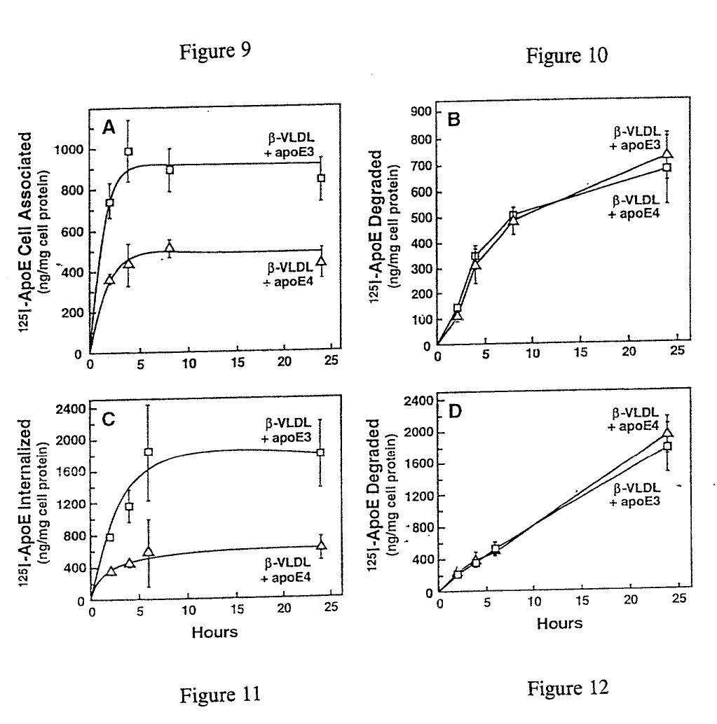

To determine if the difference in accumulation or retention of apoE3 and apoE4 by cells was due to a difference in cell association (binding and internalization) or to a difference in degradation of internalized apoE3 or apoE4, studies were performed using β-VLDL enriched with ¹²⁵I-apoE3 or ¹²⁵I-apoE4. In these studies, the differential cellular association or internalization of the iodinated apoE3 and apoE4 in both Neuro-2a cells (FIG. 9) and human skin fibroblasts (FIG. 11) was also apparent beginning at the earliest time point (2 h) and continuing to the end of the experiment (24 h). The difference in apoE3 and apoE4 content of the cells was maximal after 4 to 8 h of incubation. In the Neuro-2a cells, the amount of apoE3 associated with the cells was twice the amount of apoE4 associated with the cells (FIG. 9), whereas in fibroblasts apoE3 was threefold more abundant than apoE4 in the cells (FIG. 11). Likewise, ¹²⁵I-apoE2 also accumulated intracellularly to a greater extent than apoE4 (˜1.5-fold greater than apoE4 at 2 h). In contrast to the differential cell association or internalization of ¹²⁵I-apoE3 and ¹²⁵I-apoE4 in the Neuro-2a cells and fibroblasts, respectively, there was no significant difference in the degradation of the iodinated apoE3 or apoE4 by the cells (FIGS. 10 and 12).

The differential cellular accumulation of apoE3 and apoE4 from apoE-enriched β-VLDL was also observed in hepatocytes. As shown in Table 6, HepG2 cells incubated with ¹²⁵I-apoE3 plus β-VLDL displayed about 2.5-fold greater cell association of apoE compared with cells incubated with ¹²⁵I-apoE4 plus β-VLDL. Data from the immunological and autoradiographic studies, as well as the binding and degradation experiments, showed differential accumulation of apoE3 and apoE4 in Neuro-2a cells, fibroblasts, and hepatocytes incubated with apoE3-or apoE4-enriched β-VLDL.

TABLE 6
Cell association of ¹²⁵I-apoE3- or ¹²⁵I-apoE4-enriched
β-VLDL by HepG2 cells

| | ¹²⁵I-apoE3 | ¹²⁵I-apoE4 |
|---|---|---|
| Time | (ng/mg cell protein) | (ng/mg cell protein) |
| 4 hours | 1062 ± 171 | 515 ± 10 |
| 8 hours | 1466 ± 38 | 683 ± 6 |

Mean ± S.D. obtained from two independent experiments performed in duplicate.

In the experiments described thus far, the apoE3 and apoE4 were incubated with the β-VLDL at 37° C. for 1 h, and then the mixture was added to the cells. Separation of the mixture by fast-performance liquid chromatography demonstrated that ˜50% of the apoE was associated with β-VLDL particles. One possible reason for the differential accumulation might be that more apoE3 than apoE4 associates with the β-VLDL and that more apoE3 is therefore delivered to the cells. This possibility was ruled out by examining the amount of ¹²⁵I-apoE3 or ¹²⁵I-apoE4 associated with β-VLDL after isolation of apoE-enriched β-VLDL by fast-performance liquid chromatography. In fact, slightly more apoE4 than apoE3 was associated with the lipoprotein particles (7.0 versus 6.1 μg/mg of β-VLDL cholesterol). Furthermore, using the fast-performance liquid chromatography-purified ¹²⁵I-apoE-enriched β-VLDL, we demonstrated that the differential apoE accumulation occurred with apoE on the β-VLDL particles and not with lipid-free or lipid-poor apoE. The cell association was greater in Neuro-2a cells incubated with purified ¹²⁵I-apoE3-enriched β-VLDL than in those incubated with purified ¹²⁵I-apoE4-enriched β-VLDL (58 versus 39 ng/mg of cell protein at 2 h; 101 versus 65 ng/mg of cell protein at 4 h).

### EXAMPLE 6

### Mechanisms Responsible for Differential Accumulation of ApoE Isoforms

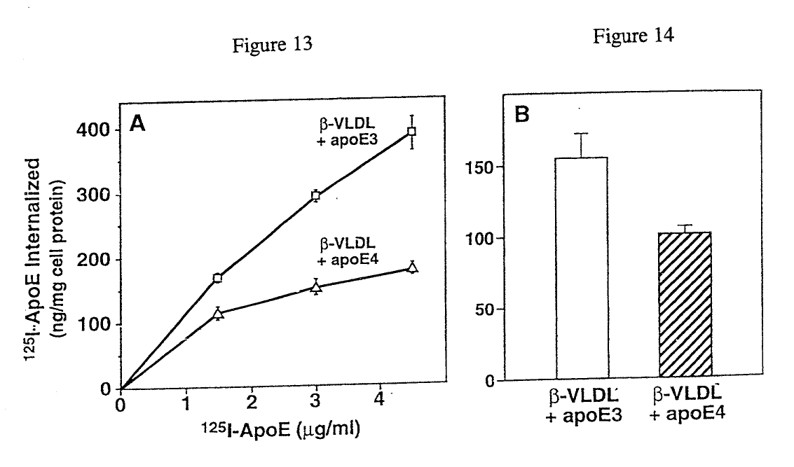

To explore in more detail how differential processing of apoE3 versus apoE4 could explain the differential accumulation, we examined the internalization of iodinated apoE-enriched β-VLDL by fibroblasts and Neuro-2a cells at 18° C., a temperature at which lipoprotein internalization occurs but degradation does not (FIGS. 13 and 14). Analysis of the culture medium for degradation products of the ¹²⁵I-apoE confirmed that degradation did not occur under the conditions used. In these studies, apoE3 accumulated to a greater extent than apoE4 in both fibroblasts (FIG. 13) and neurons (FIG. 14), demonstrating that the differential accumulation was due to differential handling of at least a portion of the internalized apoE and not to differences in lysosomal degradation. This conclusion was supported by studies in fibroblasts, in which degradation was blocked by chloroquine. Even in the absence of lysosomal degradation, the differential accumulation of apoE3 and apoE4 was apparent when the cells were incubated with apoE3- or apoE4-enriched β-VLDL.

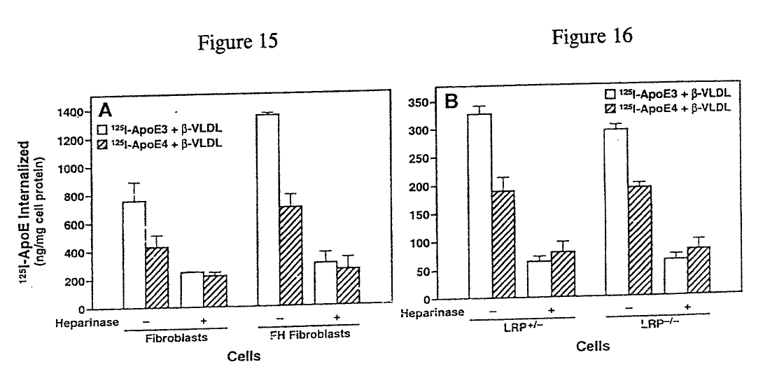

To identify the mechanism of the differential cellular accumulation of apoE3 and apoE4, we made use of fibroblasts that lacked expression of the LDL receptor, the LRP, or specific cell-surface proteoglycans. The differential cellular accumulation of the apoE3 and apoE4 from apoE-enriched β-VLDL occurred in both LDL receptor-expressing and LDL receptor-negative fibroblasts, demonstrating that the LDL receptor was not involved in the differential accumulation (FIG. 15). On the other hand, the differential accumulation was blocked totally by prior treatment of the normal or FH fibroblasts with heparinase, and the total cell association was significantly decreased for both isoforms, suggesting that the differential effect might be mediated either by the HSPG/LRP complex or by HSPG alone (FIG. 15). As shown in FIG. 16, embryonic mouse fibroblasts either heterozygous for LRP expression (LRP^{+/-}) or lacking LRP expression (LRP^{-/-}) displayed differential accumulation of apoE3 and apoE4. Therefore, LRP expression is not required for the differential accumulation of apoE3 versus apoE4. However, heparinase treatment of these cells blocked the effect, again indicating a role for cell-surface HSPG (FIG. 16). As indicated, heparinase markedly decreased total internalization of both apoE3- and apoE4-enriched β-VLDL, further suggesting the importance of HSPG alone in mediating the enhanced metabolism of apoE-enriched lipoproteins.

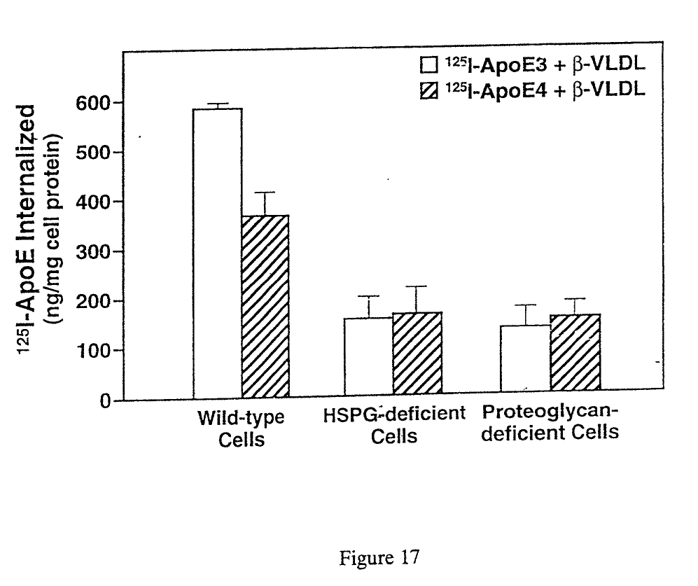

The role of HSPG in the apoE3 and apoE4 differential accumulation was examined further in control CHO cells, in mutant CHO cells specifically lacking HSPG expression, and in CHO cells lacking expression of all proteoglycans (FIG. 17). The differential cellular accumulation or retention of ¹²⁵I-apoE3 versus ¹²⁵I-apoE4 was apparent in the wild-type CHO cells; however, the differential accumulation or retention was completely abolished in both the HSPG-deficient and the proteoglycan-deficient CHO cells, conclusively demonstrating the importance of cell-surface HSPG in this process. Likewise, the levels of apoE3 and apoE4 internalized by the CHO mutant cells were very significantly reduced.

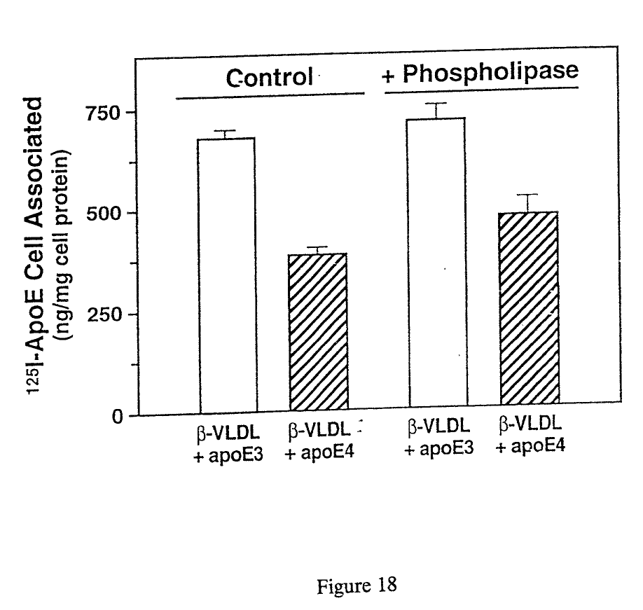

Proteoglycans associate with cell membranes either by glycerophosphatidylinositol (GPI) anchors or by transmembrane spanning of their core proteins. These classes of proteoglycans undergo different rates of cellular processing. The GPI-anchored proteoglycans exhibit fast endosome to lysosome transport and undergo lysosomal degradation with an intracellular half-life of ˜30 min, whereas the core protein-anchored proteoglycans exhibit slow endosome to lysosome transport (half-life ˜4 h) and undergo delayed processing. The retention of apoE by the cells would be consistent with use of the slow pathway for endosome to lysosome transport and would suggest that the differential accumulation of apoE3 and apoE4 in the cells is not due to internalization of apoE with GPI-anchored proteoglycans. This was demonstrated by examining the effect of specific phospholipase C, which removes GPI-anchored HSPG, on the cell association of iodinated apoE-enriched β-VLDL with fibroblasts (FIG. 18). Under the conditions used, the phospholipase removed ˜15% of ³⁵S from cells labeled for 24 h with [³⁵ S]O₄. Specific phospholipase C treatment of the cells did not affect the differential accumulation of apoE3 and apoE4 in the cells or the total binding and internalization of either the apoE3- or apoE4-enriched β-VLDL, demonstrating that GPI-anchored HSPG were not involved (FIG. 18).

Consideration was given to the possibility that the apoE4 isoform differential resulted from shunting of apoE3 specifically into an intracellular compartment and/or retroendocytosis or retarded internalization of apoE4. To evaluate these possibilities, we conducted a modified “pulse-chase” study in which CHO cells were incubated with ¹²⁵I-apoE-enriched β-VLDL for 1 h at 4° C., washed to remove unbound lipoproteins, and then warmed to 37° C. for various times to follow internalization, degradation, and retention (see Materials and Methods). At the specific times, the medium was removed for analysis of both degradation products (degraded apoE) and TCA-precipitable proteins (released intact apoE), and the cells were washed with suramin (suramin-releasable apoE) and then counted (internalized apoE).

Table 7 shows that the amount of apoE3 and apoE4 bound at 4° C. (zero time) was similar; however, the amount of apoE3 in the cells (internalized=accumulated or retained) after 30, 60, and 120 min at 37° C. was approximately twofold greater than the amount of apoE4. At each time point, we found a small amount of the ¹²⁵I-apoE that was suramin-releasable (i.e., apoE present on the cell surface). Between 30 and 120 min, the amount of ¹²⁵I-apoE3 and apoE4 degraded increased and was approximately equal for both isoforms. Thus, similar fractions of internalized apoE3 and apoE4 were degraded. Of interest was the greater amount of apoE4 that appeared in the medium during the incubation period, especially at 30 and 60 min. This TCA-precipitable, intact apoE could represent apoE that is retroendocytosed or is on or near the cell surface and rapidly released upon warming. Thus, with time apoE4 is released to a greater extent or internalized to a lesser extent than apoE3 or, alternatively, more apoE3 is sequestered into a compartment and unavailable to be released. Therefore, more apoE3 accumulates and is retained by the cells. Typically, 80-90% of the total apoE bound to the cells at 4° C. at zero time was recovered in the various fractions of the medium and cells after the warm-up periods (Table 7).

TABLE 7
Metabolism of ¹²⁵I-apoE3-and ¹²⁵I-apoE4-enriched
β-VLDL by Wild-type CHO Cells

| | 30 min | | 60 min | | 120 min | |
|---|---|---|---|---|---|---|
| | ApoE3 | ApoE4 | ApoE3 | ApoE4 | ApoE3 | ApoE4 |
| | (ng/mg cell protein) | | | | | |
| Internalized | 157 | 84 | 123 | 55 | 78 | 27 |
| (retained) | | | | | | |
| Suramin- | 45 | 27 | 39 | 10 | 12 | 10 |
| releasable | | | | | | |
| (cell surface) | | | | | | |
| Degraded | 15 | 12 | 34 | 35 | 45 | 43 |
| TCA- | 182 | 245 | 181 | 238 | 232 | 225 |
| precipitable | | | | | | |
| (release intact) | | | | | | |
| Total | 399 | 368 | 377 | 338 | 367 | 305 |

Similar amounts of ¹²⁵I-apoE3 and ¹²⁵ I-apoE4 (399 ng/mg and 378 ng/mg of cell protein, respectively) were bound to the cells at 4° C. (i.e., zero time). Recovery of ¹²⁵ I-apoE (total) in the fractions analyzed after warming to 37° C. is also reported in the table. Data represent results from one experiment performed in quadruplicate. The experiment was repeated three times with similar results.

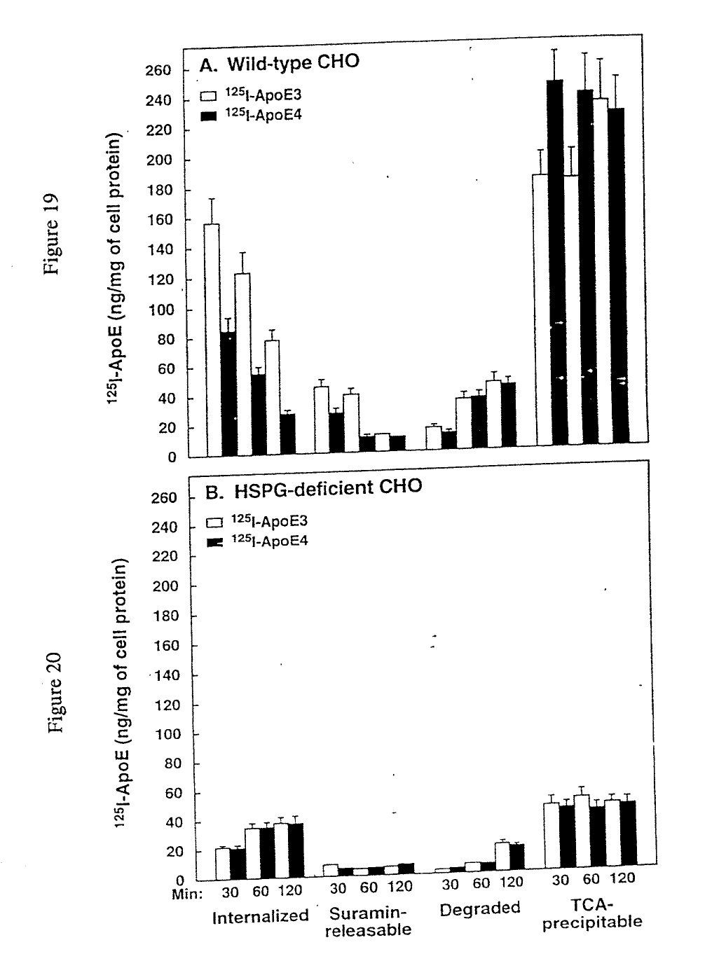

Data from this pulse-chase study are graphically illustrated in FIG. 19. Three separate experiments were performed with this design and yielded comparable results. In wild-type CHO cells, apoE3 accumulated and was retained to a greater extent than apoE4, similar amounts of apoE3 and apoE4 were degraded at all time points, and more apoE4 reappeared in the medium at 30 and 60 min. By contrast, HSPG-deficient CHO cells bound much less ¹²⁵I-apoE3 +β-VLDL and ¹²⁵I-apoE4+β-VLDL (77 and 75 ng/mg of cell protein) than wild-type CHO cells (399 and 378 ng/mg of cell protein); the HSPG-deficient cells internalized and degraded similar amounts of apoE3 and apoE4 at all time points. We also found similar amounts of suramin-releasable and TCA-precipitable ¹²⁵I-apoE3 and ¹²⁵I-apoE4 (FIG. 10B). Thus, HSPG-deficient cells not only have markedly reduced uptake of apoE but also do not show any isoform-specific differential accumulation, degradation, or retention.

The metabolism of apoE-enriched β-VLDL was examined to determine if apoE3 and apoE4 stimulate the same level of uptake of β-VLDL particles. Further, the cellular uptake (retention or accumulation) or the apoE from apoE-enriched β-VLDL is examined more directly by immunocytochemistry and by following the metabolism of iodinated apoE.

Incubation of Neuro-2a cells with either apoE3- or apoE4-enriched β-VLDL resulted in a similar cell association of β-VLDL and a similar increase of cellular cholesterol. This shows that in neurons, as in fibroblasts, apoE3 and apoE4 stimulate the uptake of similar numbers of lipoprotein particles. On the other hand, when the cellular accumulation specifically of apoE3 and apoE4 was examined in Neuro-2a cells by either immunofluorescence or analysis of extracted cellular proteins, a differential accumulation of apoE3 and apoE4 was observed. These observations were confirmed in Neuro-2a cells and extended to fibroblasts and hepatocytes by examining the cellular association of internalization of ¹²⁵I-apoE3- or ¹²⁵I-apoE4-enriched β-VLDL. In all three cell types, intracellular apoE3 accumulated to a greater extent than apoE4 (˜2-fold). Likewise, apoE2 also accumulated to a greater extent than apoE4 in Neuro-2a cells (˜1.5-fold). The differential accumulation of apoE3 and apoE4 occurred in both LDL receptor-negative human fibroblasts and in LRP-negative murine embryonic fibroblasts, demonstrating that these receptors are not significantly involved. However, the differential accumulation or retention was abolished by treating the cells with heparinase.

The role of the HSPG in this process was confirmed by the use of mutant CHO cells deficient in HSPG synthesis. In these cells, the accumulation of both apoE3 and apoE4 was reduced, and the differential accumulation of apoE3 and apoE4 was abolished. Treatment of the cells with specific phospholipase C, which releases phospholipid-anchored HSPG, had no effect on the differential accumulation of apoE3 and apoE4 from apoE-enriched β-VLDL. Enhanced degradation of apoE4 was not the reason for the difference in cellular accumulation of apoE3 and apoE4 by the cells, since the differential accumulation occurred at 18° C., a temperature at which endosome-lysosome fusion does not occur, as well as in the presence of chloroquine, which inhibits lysosomal degradation.

The pulse-chase studies (Table 7, FIGS. 19 and 20) suggest a possible mechanism for the differential accumulation or retention of apoE. After similar amounts of ¹²⁵I-apoE3- and ¹²⁵I-apoE4-enriched β-VLDL were bound to the CHO cells at 4° C., warming the cells to 37° C. resulted in internalization of more apoE3 than of apoE4. On the other hand, more apoE4 was found in the medium at the early time points (30 and 60 min) suggesting that the differential apoE accumulation and retention resulted from a preferential release of apoE4 from the cells. In these same studies, the HSPG-deficient CHO cells bound, internalized, and degraded much less apoE, and there was no differential between apoE3 and apoE4.

Cell-surface HSPG bind a number of biologically important molecules. In addition, HSPG can function as a receptor directly involved in binding and internalization of specific ligands. This has been demonstrated for certain viruses, thrombospondin, lipoprotein and hepatic lipases, thrombin, and fibroblast growth factor (FGF). In addition, HSPG facilitates the interaction of ligands with other receptors or serve as a bridge functioning like a co-receptor. For example, HSPG can facilitate the interaction of FGF with the FGF receptor, a co-receptor function for HSPG and the LRP in the binding and internalization of apoE-and hepatic lipase-containing lipoproteins. As demonstrated in the present study, apoE-containing lipoproteins can be bound and apoE internalized in an HSPG-dependent process without participation of the LDL receptor or the LRP. Heparinase treatment alone abolishes the differential accumulation of apoE. Heparinase treatment of cultured cells does not interfere with LDL receptor-mediated LDL binding or LRP-mediated binding of α₂-macroglobulin.

The ability of HSPG alone or in complex with a co-receptor to function in the internalization of ligands suggests ways in which the intracellular processing of these molecules may differ. The intracellular fate of FGF is determined by which pathway is used. When FGF is internalized by HSPG alone, it is degraded; however, when FGF is internalized via the HSPG/FGF receptor pathway, a portion of the FGF enters the cytoplasm and ultimately the nucleus. Clearly, apoE-enriched lipoproteins can be internalized by three cellular mechanisms: the LDL receptor, the HSPG/LRP pathway, and an HSPG-dependent/LRP-independent pathway. Thus, the intracellular fate of apoE may depend on the proportion of the protein entering the cell via each of these pathways. Specifically, the HSPG-dependent/LRP-independent pathway accounts for the differential handling of apoE3 versus apoE4 that is responsible for the greater accumulation of apoE3 than apoE4. One can speculate that apoE3-enriched enriched lipoprotein uptake via the HSPG pathway directs apoE3 to a separate (intracellularly sequestered) pool, allowing it to accumulate in the cells. On the other hand, apoE4-enriched lipoproteins taken up via the HSPG pathway may fail to escape the typical endosomal/lysosomal cascade and thus apoE4 does not accumulate. Alternatively, apoE4 complexed to HSPG may be recycled and released at the cell surface (retroendocytosis).

Results provided here show that incubation of neurons, fibroblasts, and hepatocytes with β-VLDL together with either apoE3 or apoE4 results in the retention of intact apoE by the cells and in a greater cellular accumulation of apoE3 than apoE4. Cell-surface HSPG appear to play a primary role in both the retention and the apoE and the differential accumulation of apoE3 versus apoE4. The LRP and the LDL receptor are not primarily involved. The intracellular fate of the apoE remains to be determined; however, the retention of apoE by the cells is most likely due to association with the slow endosome to lysosome transport of HSPG. It remains to be determined whether or not apoE in this pathway can escape lysosomal degradation and enter the cytoplasmic compartment, where it might interact with microtubule-associated proteins or other cellular components that could account for the differential effects of apoE3 and apoE4 on neurite outgrowth and the cytoskeleton.

### EXAMPLE 7

### Identification of Compounds That Interfere with Domain Interactions

We sought to identify small organic molecules that block the domain interaction in ApoE4 and reverse the enhanced risk associated with this isoform. Our strategy was to use available structural information to narrow the choices for physical testing. The recently determined structure of the N-terminal domain of human apoE4 provided an exciting opportunity for structure-based drug design. The general approach was to find molecules which bind to the appropriate region of the N-terminal domain and block the interaction with the C-terminal domain, a “negative image” approach. The Available Chemicals Directory (ACD; Molecular Design Limited, Inc., San Leandro, Calif.) has been screened computationally using the structure of the N-terminal domain of human apoE4. The ACD contains model-built coordinates of over 200,000 compounds available from chemical suppliers.

Search Methods-Negative Image Approach

In the negative image approach, the program DOCK models the binding of each candidate molecule to the target protein. Kuntz, I. D. (1992) Science 257;1078-82; and Ewing and Kuntz (1997) J Comput. Chem. 18:1175-1189. The space available for binding is described by a set of spheres that collectively fill the site. The centers of the spheres are then treated as possible ligand atom positions, and each molecule is combinatorially placed in the site in hundreds to thousands of positions. Simple scoring functions, one reflecting shape complementarity and another consisting of a Lennard-Jones van der Waals term and a Coulombic electrostatic term, are used to evaluate the positions. Precalculated grids allow rapid scoring. Meng et al. (1992) J. Comput. Chem. 13:505-524. For each molecule, the best position according to each scoring function is saved. At the end of the process, the several hundred best-scoring molecules according to each function are examined graphically. Kuntz and coworkers have applied the DOCK strategy to several targets, including the HIV1 protease and thymidylate synthase.

DOCK Search

DOCK version 4.0 was used to search the ACD against the N-terminal domain structures of both apoE3 and ApoE4. Kuntz (1997) J Comput. Chem. 18:1175-1189. The site of interest included residues 109, 112, and 61, plus surrounding regions. All protein atoms in the structure were used in computing scores. Searches were performed at two different levels of sampling (roughly, this corresponds to how many positions are tried for each molecule).

Over 2000 molecules that scored well when docked to apoE4 were output from DOCK. In most cases, molecules that also appeared on the corresponding lists for apoE3 were removed from consideration. Compounds were further screened visually using the graphics program MIDAS, by evaluation of complementarity with the target site and the presence of desired druglike characteristics. Ferrin et al. (1988). J Mol. Graph. 6:13-27; and Lipinski et al. (1997) Adv. Drug Delivery Rev. 23:3-25. For example, molecules that were too large, hydrophobic, or peptide-like were removed from consideration. Natural products with a large number of stereocenters were also discarded, as they would not be amenable to synthesis of derivatives. This process led to a list of 115 compounds, with 65 initial recommendations (one per set of close analogs).

Assay for Domain Interaction

Since apoE4 displays a preference for large triglyceride-rich lipoprotein particles that is mediated by domain interaction, an emulsion binding assay was developed to test the candidate compounds for their ability to interfere with domain interaction.

Preparation of emulsion particles. Triolein (160 mg) and L-alpha-Phosphatidylcholine (40 mg) are combined and dried under nitrogen. After the addition of 8 mls of buffer (10 mM Tris, 100 mM KCl, 1 mM EDTA, pH 8.0), the mixture is sonicated in a water bath to obtain a heterogeneous mix of emulsion particles. The particles are harvested by ultracentrifugation (TLA 100.2 rotor, 30,000 rpm for 30 minutes) and the subsequent lipid cake is removed by tube slicing and resuspended in 100 μl 20 mM Phosphate Buffer (PB). Triolein and phospholipid content are measured and total emulsion particle concentration is determined.

Radiolabelling. Freshly denatured and renatured Apolipoprotein E3 and E4 are radiolabelled using Bolton-Hunter Reagent [¹²⁵ I] (ICN). Specific Activity is determined using Lowry method and Gamma 8000 counter.

Binding Affinity Assay. The binding affinity of apoE3 and apoE4 to emulsion particles was determined as follows. In glass tubes, 25 μg of protein (with iodinated tracer) was reduced with 1% β-mercaptoethanol. Two hundred and fifty μg of emulsion particles and 2.5 μl of compound (10 mM stock) were added and the final mixture was brought up to 250 μl with 20 mM phosphate buffer (PB). The reaction mixture was then incubated in a 37° C. water bath for 2 hours before being transferred to 1.5 ml ultracentrifuge tubes. Finally, 50 μl of 60% sucrose was mixed with the sample and 400 μl 20 mM PB was carefully layered on top. Using a TLA 100.2 rotor, the tube was spun at 30,000 rpm for 30 minutes and subsequently cut to separate the floating emulsion particle layer from the free protein at the bottom of the tube. These fractions were then combined with the respective half of the actual tube and counted using a Gamma-8000. From these results, total emulsion-bound protein was compared to total free protein. Protein-only assays yielded 94.5-96.6% of protein accumulated in the bottom portion of the tube. In emulsion particle-only assays, 94% of emulsion particles accumulated in the top portion of the tube.

Control binding assays were conducted without the addition of compounds to determine recovery and apoE3 and apoE4 respective affinity for emulsion particles. Table 8 shows the results.

TABLE 8

| | Apo E3, n = 9 | Apo E4, n = 9 |
|---|---|---|
| | % (bound/free) | % (bound/free) |
| Mean | 29.8/70.2 | 59.4/40.6 |
| Range | 20-39/61-81 | 50-70/30-50 |
| Median | 33/67 | 60/40 |
| Mean Recovery | 92% | 88% |

Once the Apo E3 and E4 binding affinity had been determined, assays including the DOCK compounds were conducted. ApoE4 controls were included in the initial assay and apoE3 and apoE4 controls were included in the follow up assay.

In an initial screen, 14 compounds interfered with domain interaction and 6 partially interfered. In a follow-up assay, 8 of the 14 compounds were confirmed to interfere with domain interaction with little or no effect on the binding of apoE3 to the emulsions. Table 9 shows the results of the eight compounds that interfere with domain interaction. Values are provided as % bound/% free of either apoE4 (“E4”) or apoE3 (“E3).”

TABLE 9

| | | | | | | | | | |
|---|---|---|---|---|---|---|---|---|---|
| | | | | | | E4 + cpd | | E3 control | E3 + |
| Compound | Supplier | Cat. # | Family | E4 + cpd | E4 control | n = 3 | E4 control | n = 4 | compound |

| | | | | | | | | | |
|---|---|---|---|---|---|---|---|---|---|
| Z-D-Tyr (BZL)-OH | Bachem | C- | blocked | 25/75 | 69/31 | 57/43 | 43/57 | 33.5/66.5 | 26/74 |
| | | 1415 | amino acid | 23/77 | 62/38 | | | | |
| Azocarmine G | Acros | 40157- | disulfonate | 12/88 | 49/51 | 57/43 | 46/54 | 33.5/66.5 | 30/70 |
| | | 0250 | | 15/85 | 53/47 | | | | |
| Glycine cresol red | Fluka | 50100 | dye | 29/71 | 57/43 | 57/43 | 48/52 | 33.5/66.5 | 33/67 |
| | | | | 23/77 | 48/52 | | | | |
| Erythrosin B | ICN | 190450 | dye | 11/89 | 57/43 | 57/43 | 26/74 | 33.5/66.5 | 20/80 |
| | | | | 10/90 | 48/52 | | | | |
| 5-chloro-2-(4-chloro-2-(3,4- | Aldrich | S39863- | mono- | 22/78 | 57/43 | 57/43 | 49/51 | 33.5/66.5 | 14/86 |
| dichloro phenylureido | | 2 | sulfanate | 19/81 | 48/52 | | | | |
| RCL S19, 214-7 | Aldrich | S19214- | mono- | 33/67 | 57/43 | 57/43 | 48/52 | 33.5/66.5 | 29/71 |
| | | 7 | sulfoalkyl | 36/64 | 59/41 | | | | |
| | | | compound | | | | | | |
| 3-butyl-1-ethyl-5-(2-(3-sulfo- | Synthon | ST- | mono- | 28/72 | 60/40 | 57/43 | 45/55 | 33.5/66.5 | 30/70 |
| butyl-benzo(1,3)oxazo | | 342 | sulfoalkyl | 21/79 | 60/40 | | | | |
| | | | compound | | | | | | |
| RCL S3, 301-5 | Aldrich | S03301- | misc. | 19/81 | 59/41 | 57/43 | 38/62 | 33.5/66.5 | 26/74 |
| | | 5 | | 18/82 | 57/43 | | | | |

Although the foregoing invention has been described in some detail by way of illustration and example for purposes of clarity of understanding, it will be apparent to those skilled in the art that certain changes and modifications may be practiced. Therefore, the description and examples should not be construed as limiting the scope of the invention, which is delineated by the appended claims.

What is claimed is:

1. A composition comprising an agent that specifically reduces apolipoprotein E4 (apoE4) domain interaction by at least about 10%.

2. The composition according to claim 1, wherein said agent is an organic molecule having a molecular weight in the range of from about 50 daltons to about 2500 daltons.

3. The composition according to claim 1, wherein said agent inhibits formation of a salt bridge between Arg-61 and Glu-255 of apoE4.

4. A composition comprising an agent that reduces apolipoprotein E4 (apoE4) domain interaction by at least about 10%, wherein said agent is an organic molecule having a molecular weight in a range of from about 50 daltons to about 2500 daltons, and wherein said agent inhibits formation of a salt bridge between Arg-61 and Glu-255 of apoE4.

5. A method of reducing apolipoprotein E4 (apoE4) domain interaction in a cell, comprising contacting a cell that synthesizes apoE4 with an agent that reduces apoE4 domain interaction.

6. The method of claim 5, wherein said agent is an organic molecule having a molecular weight in the range of from about 50 daltons to about 2500 daltons.

7. The method according to claim 5, wherein said agent inhibits formation of a salt bridge between Arg-61 and Glu-255 of apoE4.

8. A method of promoting neuronal cell growth, comprising contacting a neuronal cell that produces apolipoprotein E4 (apoE4) or that takes up apoE4 from its environment with an agent that reduces apoE4 domain interaction, whereby neuronal cell growth is promoted.

9. The method according to claim 8, wherein said agent is an organic molecule having a molecular weight in the range of from about 50 daltons to about 2500 daltons.

10. The method according to claim 8, wherein said agent inhibits formation of a salt bridge between Arg-61 and Glu-255 of apoE4.

11. A method of promoting neuronal cell growth, comprising contacting a neuronal cell that produces apolipoprotein E4 (apoE4) or that takes up apoE4 from its environment with an agent that reduces apoE4 domain interaction, wherein said agent is an organic molecule having a molecular weight in the range of from about 50 daltons to about 2500 daltons, and wherein said agent inhibits formation of a salt bridge between Arg-61 and Glu-255 of apoE4.

12. A method of reducing formation of neurofibrillary tangles in an individual, comprising administering to the individual an effective amount of an agent that reduces apoE4 domain interaction.

13. The method according to claim 12, wherein said agent is an organic molecule having a molecular weight in the range of from about 50 daltons to about 2500 daltons.

14. A method of reducing formation of neurofibrillary tangles in an individual, comprising administering to the individual an effective amount of an agent that reduces apoE4 domain interaction, wherein said agent is an organic molecule having a molecular weight in the range of from about 50 daltons to about 2500 daltons.

15. A method for reducing the risk that an individual will develop Alzheimer's disease (AD), comprising administering to an individual at risk for developing AD an effective amount of an agent that reduces apoE4 domain interaction, wherein said agent is an organic molecule having a molecular weight in the range of from about 50 daltons to about 2500 daltons.

16. A method for reducing the severity of a symptom associated with Alzheimer's disease (AD), comprising administering to an individual who exhibits a symptom associated with AD an effective amount of an agent that reduces apoE4 domain interaction, wherein said agent is an organic molecule having a molecular weight in the range of from about 50 daltons to about 2500 daltons.

17. The method according to claim 16, wherein the symptom associated with AD is selected from the group consisting of cognitive decline and memory loss.

18. A method for reducing apolipoprotein E4 (apoE4)-mediated inhibition of neurite outgrowth, comprising contacting a neuron that synthesizes apoE4 or that takes up apoE4 from its environment with an agent that reduces apoE4 domain interaction, wherein said agent is an organic molecule having a molecular weight in the range of from about 50 daltons to about 2500 daltons.

19. A pharmaceutical formulation comprising an agent that specifically reduces apolipoprotein E4 (apoE4) domain interaction by at least about 10%, and a pharmaceutically acceptable excipient.

20. The formulation according to claim 19, wherein said agent is an organic molecule having a molecular weight in the range of from about 50 daltons to about 2500 daltons.

21. The formulation according to claim 19, wherein said agent inhibits formation of a salt bridge between Arg-61 and Glu-255 of apoE4.

22. A pharmaceutical formulation comprising an agent that reduces apolipoprotein E4 (apoE4) domain interaction by at least about 10%, wherein said agent is an organic molecule having a molecular weight in a range of from about 50 daltons to about 2500 daltons, and wherein said agent inhibits formation of a salt bridge between Arg-61 and Glu-255 of apoE4; and a pharmaceutically acceptable excipient.

23. A method of reducing apolipoprotein E4 (apoE4) domain interaction in a bodily fluid, comprising contacting a apoE4 in the fluid with an agent that reduces apoE4 domain interaction.

24. The method of claim 23, wherein the fluid is serum.

25. The method of claim 23, wherein the fluid is interstitial fluid.

26. The method of claim 23, wherein said agent is an organic molecule having a molecular weight in the range of from about 50 daltons to about 2500 daltons.

27. The method according to claim 23, wherein said agent inhibits formation of a salt bridge between Arg-61 and Glu-255 of apoE4.

28. A method of reducing apolipoprotein E4 (apoE4)-mediated inhibition of neurite outgrowth in an individual, comprising administering to the individual in need thereof an agent that reduces apoE4 domain interaction, wherein said agent is an organic molecule having a molecular weight in the range of from about 50 daltons to about 2500 daltons.
